SEQUENCE LISTING (115 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 6 aa, Homo sapiens
VQIVYK

SEQ ID NO: 3 - protein, 6 aa, Homo sapiens
VQIINK

SEQ ID NO: 4 - protein, 8 aa, synthetic construct
  VARIANT 2: note = X denotes L or F
  VARIANT 5: note = X denotes K or E
  VARIANT 7: note = X denotes D or Y
  VARIANT 8: note = X denotes Y or F
GXNIXDXX

SEQ ID NO: 5 - protein, 8 aa, synthetic construct
  VARIANT 6: note = X denotes G or D
  VARIANT 7: note = X denotes D, E, or K
  VARIANT 8: note = X denotes P, T, or N
IDPEBXXX

SEQ ID NO: 6 - protein, 7 aa, synthetic construct
  VARIANT 1: note = X denotes F or no amino acid
XIRDFEV

SEQ ID NO: 7 - protein, 7 aa, synthetic construct
  VARIANT 3: note = X denotes V or I
  VARIANT 6: note = X denotes S or T
  VARIANT 7: note = X denotes Y or F
SSXSSXX

SEQ ID NO: 8 - protein, 11 aa, synthetic construct
  VARIANT 2: note = X denotes S or N
  VARIANT 6: note = X denotes G or D
  VARIANT 7: note = X denotes S or N
QXIVHXXGHTY

SEQ ID NO: 9 - protein, 8 aa, synthetic construct
  VARIANT 1: note = X denotes H or F
  VARIANT 3: note = X denotes Y or G
  VARIANT 4: note = X denotes H, S, or R
  VARIANT 5: note = X denotes S, V, or I
XQXXXPLT

SEQ ID NO: 10 - protein, 8 aa, synthetic construct
  VARIANT 2: note = X denotes Y or F
  VARIANT 4: note = X denotes F or G
  VARIANT 5: note = X denotes S or T
  VARIANT 6: note = X denotes S, N or T
GXTXXXYG

SEQ ID NO: 11 - protein, 11 aa, synthetic construct
  VARIANT 1: note = X denotes A, G or V
  VARIANT 4: note = X denotes R or V
  VARIANT 5: note = X denotes T, P, or N
  VARIANT 8: note = X denotes F or Y
XRWXXGYXFDY

SEQ ID NO: 12 - protein, 10 aa, synthetic construct
SSLWETWLAY

SEQ ID NO: 13 - protein, 11 aa, synthetic construct
  VARIANT 1: note = X denotes Q, R or L
  VARIANT 2: note = X denotes N, T or S
  VARIANT 4: note = X denotes V or I
  VARIANT 6: note = X denotes S or T
XXIXHXNGBTY

SEQ ID NO: 14 - protein, 9 aa, synthetic construct
  VARIANT 3: note = X denotes S, T, or G
  VARIANT 5: note = X denotes L or H
  VARIANT 6: note = X denotes V or I
  VARIANT 8: note = X denotes L or P
FQXSXXPXT

SEQ ID NO: 15 - protein, 15 aa, synthetic construct
QIVYKPVDLSKVTSC

SEQ ID NO: 16 - protein, 15 aa, synthetic construct
QIINKKLDLSNVQSC

SEQ ID NO: 17 - protein, 15 aa, synthetic construct
  MOD_RES 5: note = Acetylation
QIINKKLDLSNVQSC

SEQ ID NO: 18 - protein, 112 aa, synthetic construct
EVQLQQSGAEFVRPGASVKLSCTVSGLNIEDDYIQWLKQRPEQGLEWIGWIDPENGDPKYASKFQGKATLTADTSSKIAYLHLSSLTSEDTAVYYCATRGLWGQGTTLTVSS

SEQ ID NO: 19 - protein, 108 aa, synthetic construct
QIVLTQSPAIMSASLGERVTMTCTASSSVSSSYFHWYQQKPGSSPKLWIYSTSNLASGVPVRFSGSGSGTSYSLTISSMEAEDAATYYCHQYHRSPLTFGAGTKLELK

SEQ ID NO: 20 - protein, 112 aa, synthetic construct; identical to SEQ ID NO: 18

SEQ ID NO: 21 - protein, 108 aa, synthetic construct; identical to SEQ ID NO: 19

SEQ ID NO: 22 - protein, 112 aa, synthetic construct; identical to SEQ ID NO: 18

SEQ ID NO: 23 - protein, 108 aa, synthetic construct; identical to SEQ ID NO: 19

SEQ ID NO: 24 - protein, 112 aa, synthetic construct; identical to SEQ ID NO: 18

SEQ ID NO: 25 - protein, 108 aa, synthetic construct
QIVLTQSPPIMSASLGERVTMTCTASSSVSSSYFHWYQQKPGSSPKLWIYSTSNLASGVPVRFSGSGSGTSYSLTISTMEAEDAATYYCHQYHRSPLTFGAGTKLELK

SEQ ID NO: 26 - protein, 113 aa, synthetic construct
EVRLQQSGAEFVKPGASVKLSCTSSGFNIKDYYMHWVKQRTEQGLEWIGMIDPENGETKYAPKFQGKATITADTSSNTAYLHLSSLTSEDTAVYYFIRDFEVWGTGTTVTVSS

SEQ ID NO: 27 - protein, 112 aa, synthetic construct
DVLMTQTPLSLPVSLGDQASISCRSSQNIVHGSGHTYLEWYLQMPGQSPKLLIHKVSNRLSGVPDRFSGSGSGTDFTLKISRVEAEDLGVYYCFQGSHVPLTFGAGTKLELR

SEQ ID NO: 28 - protein, 112 aa, synthetic construct
EVQLQQSGAELVRPGASVKLSCTASGLNIKDDYMQWIKQRPEQGLEWIGWIDPENGDTKYASKFQGKATITADTSSNTAYLQLSSLTSEDTAVYFCATRGLWGQGTTLTVSS

SEQ ID NO: 29 - protein, 108 aa, synthetic construct
QIVLTQSPAIMSASLGERVTMTCTASSSVSSSYFHWYQQKPGSSPKLWIYSTSNLASGVPARFSGSGSGTSYSLTISSMEAEDAATYYCHQYHRSPLTFGAGTKLELK

SEQ ID NO: 30 - protein, 112 aa, synthetic construct
EVQLQQSGAELVRPGASVKLSCTTSGLNIKDDYVNWVKQRPEQGLEWIGWIDPENDDTKYASKFQGKATITAATSSSTAYLHLNSLTSEDSAVYYCATRGVWGQGTTLTVSS

SEQ ID NO: 31 - protein, 108 aa, synthetic construct
QIVLTQSPAIMSASLGERVTMTCTASSSISSTFFHWYQQKTGSSPKLWIYRTSNLAPGVPARFSGSGSGTSYSLTISNMEAEDTATYYCHQYHRSPLTFGAGTKLELK

SEQ ID NO: 32 - protein, 113 aa, synthetic construct
EVQLQQSGAELVKPGASVKLSCTTSGFNIKDYFMHWVKQRTEQGLEWIGMIDPEDGKTKYAPKFQGKATITADPSSNTAYLHLSSLTSEDTAVYYIIRDFEVWGTGTTVTVSS

SEQ ID NO: 33 - protein, 112 aa, synthetic construct
DVLMTQTPLSLPVSLGDQASISCRSSQSIVHDNGHTYLEWYLQMPGQSPKLLIHKVSNRLSGVPDRFSGSGSGTDFTLKINRVEAEDLGVYYCFQGSHIPLTFGAGTKLELK

SEQ ID NO: 34 - protein, 113 aa, synthetic construct; identical to SEQ ID NO: 26

SEQ ID NO: 35 - protein, 112 aa, synthetic construct; identical to SEQ ID NO: 27

SEQ ID NO: 36 - protein, 112 aa, synthetic construct
EVQLQQSGAEFVRPGASVKLSCTVSGLNIEDDYIQWLKQRPEQGLEWIGWIDPENGDPNYASKFQGKATLTADTSSKIAYLHLSSLTSEDTAVYYCATRGLWGQGTTLTVSS

SEQ ID NO: 37 - protein, 108 aa, synthetic construct; identical to SEQ ID NO: 19

SEQ ID NO: 38 - protein, 112 aa, synthetic construct; identical to SEQ ID NO: 18

SEQ ID NO: 39 - protein, 108 aa, synthetic construct; identical to SEQ ID NO: 19

SEQ ID NO: 40 - protein, 112 aa, synthetic construct
EVQLQQSGAELVRPGASVKLSCTVSGLNIKDDYIHWVKQRPEQGLEWIGWIDPENGDNEYASKFQGKATITADTSSKTAYLQFSSLTSEDTAVYYCTTRGLWGQGTTLTVSS

SEQ ID NO: 41 - protein, 108 aa, synthetic construct
QIVLTQSPAIMSASLGERVTMTCTASSSVSSSYLLWFQQRPGSSPKLWIYSTSNLASGVPARFSGSGSGTSYSLTISSMEAEDAATYYCHQYRRSPLTFGAGTKLELK

SEQ ID NO: 42 - protein, 113 aa, synthetic construct; identical to SEQ ID NO: 26

SEQ ID NO: 43 - protein, 112 aa, synthetic construct; identical to SEQ ID NO: 27

SEQ ID NO: 44 - protein, 118 aa, synthetic construct
EVQLQQSGAELVRPGSSVKMSCKTSGYTFTNYGLNWVKQRPGQGLEWIGYIYIGNGYTDYNENFKGKATLTSDTSSSTAYMQLSSLTSEDSAIYFCARWRPGYFFDYWGQGTTLTVSS

SEQ ID NO: 45 - protein, 112 aa, synthetic construct
DVLMTQTPLSLPVGLGDQASISCRSSQNIVHSNGDTYLEWYLLKPGQSPQLLIYKVSNRFSGVPDRFSGGGSGTDFILKISRVEAEDLGVYYCFQSSLVPLTFGAGTKLELK

SEQ ID NO: 46 - protein, 117 aa, synthetic construct
EVQLQQSGAELVRPGSSVKMSCKTSGYTFTNYGLNWVKQRPGQGLEWIGYIYIGNGYTDYNENFKGKATLTSDTSSSTAYMQLSSLTSEDSAIYFCARWRPGYFFDYWGQGTTLTVS

SEQ ID NO: 47 - protein, 112 aa, synthetic construct
DVLMTQTPLSLPVSLGDQASISCRSSQNIVHTNGDTYLEWYLLKPGQSPQLLIYKVSNRFSGVPDRFSGGGSGTDFTLKISRVEAEDLGVYFCFQSSLVPLTFGAGTKLELK

SEQ ID NO: 48 - protein, 118 aa, synthetic construct
EVQLQQSGAELVRPGSSVKMSCKTSGYTFTTYGLNWVKQRPGQGLEWIGYIYFGNGYTDYSEKFKGKATLTSDTSSSTAYMQLSSLTSEDSAIYFCARWVTGYYFDYWGQGTTLTVSS

SEQ ID NO: 49 - protein, 111 aa, synthetic construct
DVLMTQIPLSLPVSLGDQASISCRSSRTIVHSNGNTYLEWYLQKPGQSPKLLIYKVSNRFSGVPDRFSGSGSGTDFTLKISRVEAEDLGVYYCFQSSLVPPTFGGGTKLEI

SEQ ID NO: 50 - protein, 118 aa, synthetic construct; identical to SEQ ID NO: 44

SEQ ID NO: 51 - protein, 112 aa, synthetic construct
DVLMTQTPLSLPVGLGDQASISCRSSQNIVHSNGDTYLEWYLLKPGQSPQLLIYKVSNRFSGVPDRFSGGGSGTDFTLKISRVEAEDLGVYYCFQSSLVPLTFGAGTKLELK

SEQ ID NO: 52 - protein, 118 aa, synthetic construct
EVQLQQSGAELVRPGSSVKMSCKTSGYTFTNYGVIWVKQRPGQGLEWIGYIYIGNGFTDYSEKFKGKATLTSDTSSSTAYMHLISLTSEDSAIYFCGRWRPGYYFDYWGQGTSLTVSS

SEQ ID NO: 53 - protein, 112 aa, synthetic construct
DVLMTQIPLSLPVSLGDQASISCRSSQNIVHSNGNTYLEWYLQKPGQSPKLLIYKVSNRFSGVPDRFSGSGSGTDFTLKISRVEAEDLGVYYCFQSSLVPLTFGAGTKLELK

SEQ ID NO: 54 - protein, 118 aa, synthetic construct
EVQLQQSGVELVRPGSSVKMSCKTSGYTFTNYGIIWVKQRPGQGLEWIGYIYIGNGYTDCNEKFKGKASLTSDTSSSTAYMHLSSLTSEDSAIYFCGRWRPGYYFDYWGQGTTLTVSS

SEQ ID NO: 55 - protein, 112 aa, synthetic construct
NVLMTQTPLSLPVSLGDQASISCRSSQTIVHSNGNTYLEWYLQKPGQSPKLLIYKVSNRFSGVPERFSGSGSGTDFTLKISRVEAEDLGVYYCFQSSLVPLTFGAGTKLELK

SEQ ID NO: 56 - protein, 118 aa, synthetic construct
EVQLQQSGVELVRPGSSVKMSCKTSGYTFTNYGIIWVKQRPGQGLEWIGYISIGNGYTDCNEKFKGKASLTSDTSSSTAYMHLSSLTSEDSAIYFCGRWRPGYYFDYWGQGTTLTVSS

SEQ ID NO: 57 - protein, 112 aa, synthetic construct
DVLMTQTPLSLPVSLGDQASISCRSSQTIVHSNGNTYLEWYLQKPGQSPKLLIYKVSNRFSGVPERFSGSGSGTDFTLKISRVEAEDLGVYYCFQSSLVPLTFGAGTKLELK

SEQ ID NO: 58 - protein, 118 aa, synthetic construct
EVQLQQSGAELVRPGSSVQMSCKTSGYTFTNYGINWVRQRPGQGLEWIGYIYIGSGYTDYKEKFRGKATLTSDTSSSTAYMQLSSLTSEDSAIYFCARWVNGYYFDYWGQGTTLAVSS

SEQ ID NO: 59 - protein, 111 aa, synthetic construct
DVLMTQTPLSLPVSLGDQASISCRSSQSIIHSNGNTYLEWYLQKPGQSPKLLIYKVSHRFSGVPDRFSGSGSGTDFSLKISRVEAEDLGVYYCFQSSLVPPTFGGGTKLEI

SEQ ID NO: 60 - protein, 118 aa, synthetic construct; identical to SEQ ID NO: 54

SEQ ID NO: 61 - protein, 112 aa, synthetic construct; identical to SEQ ID NO: 57

SEQ ID NO: 62 - protein, 118 aa, synthetic construct
EVQFQQSGAELVRPGSSVKMSCKTSGYTFTSYGINWVKQRPGQGLEWIGYIYIGNGYTDYNEKFRGKATLTSDTSSSTAYMQLSGLTSEDSATYFCVRWRPGYFFDYWGQGTTLTVSS

SEQ ID NO: 63 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVETEDLGVYYCFQTSLVPLTFGAGTRLELK

SEQ ID NO: 64 - protein, 117 aa, synthetic construct
EVQLVESGGDLVKPGGSLKVSCAASGFTFSSYGMSWIRQTPDNTLEWVATISYGGTYTFYSDNVKGRFTISRDDAKNTLYLQMSSLKSEDTAMYYCSSLWETWLAYWGQGTLVTVSA

SEQ ID NO: 65 - protein, 111 aa, synthetic construct
DVLMTQTPLSLPVSLGDQASISCRSSLSIVHSNGNTYLEWYLQKPGQSPKLLIYRVSNRFSGVPDRFSGSGSGTDFTLKISRVEAEDLGVYYCFQGSHIPPTFGGGTKLEI

SEQ ID NO: 66 - protein, 8 aa, synthetic construct
GLNIEDDY

SEQ ID NO: 67 - protein, 8 aa, synthetic construct
GFNIKDYY

SEQ ID NO: 68 - protein, 8 aa, synthetic construct
GLNIKDDY

SEQ ID NO: 69 - protein, 8 aa, synthetic construct
GFNIKDYF

SEQ ID NO: 70 - protein, 8 aa, synthetic construct
IDPENGDP

SEQ ID NO: 71 - protein, 8 aa, synthetic construct
IDPENGET

SEQ ID NO: 72 - protein, 8 aa, synthetic construct
IDPENGDT

SEQ ID NO: 73 - protein, 8 aa, synthetic construct
IDPENDDT

SEQ ID NO: 74 - protein, 8 aa, synthetic construct
IDPEDGKT

SEQ ID NO: 75 - protein, 8 aa, synthetic construct
IDPENGDN

SEQ ID NO: 76 - protein, 5 aa, synthetic construct
ATRGL

SEQ ID NO: 77 - protein, 6 aa, synthetic construct
IRDFEV

SEQ ID NO: 78 - protein, 5 aa, synthetic construct
ATRGV

SEQ ID NO: 79 - protein, 5 aa, synthetic construct
TTRGL

SEQ ID NO: 80 - protein, 7 aa, synthetic construct
FIRDFEV

SEQ ID NO: 81 - protein, 7 aa, synthetic construct
SSVSSSY

SEQ ID NO: 82 - protein, 11 aa, synthetic construct
QNIVHGSGHTY

SEQ ID NO: 83 - protein, 7 aa, synthetic construct
SSISSTF

SEQ ID NO: 84 - protein, 11 aa, synthetic construct
QSIVHDNGHTY

SEQ ID NO: 85 - protein, 9 aa, synthetic construct
HQYHRSPLT

SEQ ID NO: 86 - protein, 9 aa, synthetic construct
FQGSHVPLT

SEQ ID NO: 87 - protein, 9 aa, synthetic construct
FQGSHIPLT

SEQ ID NO: 88 - protein, 9 aa, synthetic construct
HQYRRSPLT

SEQ ID NO: 89 - protein, 8 aa, synthetic construct
GYTFTNYG

SEQ ID NO: 90 - protein, 8 aa, synthetic construct
GYTFTTYG

SEQ ID NO: 91 - protein, 8 aa, synthetic construct
GYTFTSYG

SEQ ID NO: 92 - protein, 8 aa, synthetic construct
GFTGSSYG

SEQ ID NO: 93 - protein, 8 aa, synthetic construct
IYIGNGYT

SEQ ID NO: 94 - protein, 8 aa, synthetic construct
IYFGNGYT

SEQ ID NO: 95 - protein, 8 aa, synthetic construct
IYIGNGFT

SEQ ID NO: 96 - protein, 8 aa, synthetic construct
ISIGNGYT

SEQ ID NO: 97 - protein, 8 aa, synthetic construct
IYIGSGYT

SEQ ID NO: 98 - protein, 8 aa, synthetic construct
ISYGGTYT

SEQ ID NO: 99 - protein, 11 aa, synthetic construct
ARWRPGYFFDY

SEQ ID NO: 100 - protein, 11 aa, synthetic construct
ARWVTGYYFDY

SEQ ID NO: 101 - protein, 11 aa, synthetic construct
GRWRPGYYFDY

SEQ ID NO: 102 - protein, 11 aa, synthetic construct
ARWVNGYYFDY

SEQ ID NO: 103 - protein, 11 aa, synthetic construct
VRWRPGYFFDY

SEQ ID NO: 104 - protein, 10 aa, synthetic construct
SSLWETWLAY

SEQ ID NO: 105 - protein, 11 aa, synthetic construct
QNIVHSNGDTY

SEQ ID NO: 106 - protein, 11 aa, synthetic construct
QNIVHTNGDTY

SEQ ID NO: 107 - protein, 11 aa, synthetic construct
RTIVHSNGNTY

SEQ ID NO: 108 - protein, 11 aa, synthetic construct
QTIVHSNGNTY

SEQ ID NO: 109 - protein, 11 aa, synthetic construct
QSIIHSNGNTY

SEQ ID NO: 110 - protein, 11 aa, synthetic construct
QSIVHSNGNTY

SEQ ID NO: 111 - protein, 11 aa, synthetic construct
LSIVHSNGNTY

SEQ ID NO: 112 - protein, 9 aa, synthetic construct
FQSSLVPLT

SEQ ID NO: 113 - protein, 9 aa, synthetic construct
FQSSLVPPT

SEQ ID NO: 114 - protein, 9 aa, synthetic construct
FQTSLVPLT

SEQ ID NO: 115 - protein, 9 aa, synthetic construct
FQGSHIPPT

(19) United States
(12) Patent Application Publication
(10) Pub. No.: US 2024/0376188 A1
(43) Pub. Date: Nov. 14, 2024

(54) MONOCLONAL ANTIBODIES TARGETING ACETYLATED TAU AND METHODS OF USE THEREOF

(71) Applicant: The University of North Carolina at Chapel Hill, Chapel Hill, NC (US)

(72) Inventors: Miles Bryan, Durham, NC (US); Todd Cohen, Chapel Hill, NC (US)

(21) Appl. No.: 18/690,409

(22) PCT Filed: Sep. 8, 2022
(86) PCT No.: PCT/US2022/076088

§ 371 Date: Mar. 8, 2024

Related U.S. Application Data
(60) Provisional application No. 63/242,083, filed on Sep. 9, 2021.

(51) Int. Cl. C07K 16/18; G01N 33/68

(52) U.S. Cl. CPC C07K 16/18; G01N 33/6896; C07K 2317/24; C07K 2317/565; C07K 2317/77; G01N 2440/10; G01N 2800/2821

(57) ABSTRACT

The invention relates to monoclonal antibodies or an antigen binding fragment thereof targeting acetylation sites within the human Tau protein. K280 and K311, which can be indicative of a disease state and, as such, represent diagnostic and/or therapeutic targets. Accordingly, one aspect of the invention relates to a monoclonal antibody or an antigen binding fragment thereof that specifically binds acetylated lysine 280 or acetylated lysine 311 in human tau protein. The monoclonal antibody or an antigen binding fragment thereof can be part of a pharmaceutical composition and provided to a subject to diagnose and/or treat a tauopathy.

### STATEMENT OF PRIORITY

This application claims the benefit, under 35 U.S.C. § 119 (e), of U.S. Provisional Application No. 63/242,083, filed Sep. 9, 2022, the entire contents of which is incorporated by reference herein in its entirety.

### STATEMENT OF GOVERNMENT SUPPORT

This invention was supported in part by funding under Government Grant No. 1F32AG072826-01, awarded by the National Institutes of Health. The United States Government has certain rights in this invention.

### FIELD OF THE INVENTION

The invention relates to monoclonal antibodies or an antigen binding fragment thereof targeting acetylation sites within the human Tau protein, K280 and K311, which are indicative of a disease state and, as such, represent diagnostic and/or therapeutic targets. Accordingly, one aspect of the invention relates to a monoclonal antibody or an antigen binding fragment thereof that specifically binds acetylated lysine 280 or acetylated lysine 311 in human tau protein. The monoclonal antibody or an antigen binding fragment thereof can be part of a pharmaceutical composition and provided to a subject to diagnose and/or treat a tauopathy.

### BACKGROUND OF THE INVENTION

Alzheimer's disease (AD) is an age-related, progressive neurodegenerative disease without effective disease-modifying therapies. AD is the most common and expensive form of neurodegeneration in the US, accounting for a total of 5.5 million patients. As average life-expectancy increases, the baby boomer generation is projected to double the total number of people with AD in the United States up to 10 million, and likely triple cases worldwide. This calls for a desperate need for new diagnostic and therapeutic approaches to AD. AD is characterized and diagnosed by the formation of both insoluble intracellular neurofibrillary tangles of the microtubule associated protein tau (MAPT) and extracellular amyloid beta (AB) plaques. Unlike AB, tau accumulation has been shown to be strongly associated with disease progression and symptomology suggesting tau might drive AD progression. As suppression of total tau may lead to undesirable effects on tau-mediated microtubule (MT) stabilization, many have turned to targeting specific tau strains. A plethora of previous studies have discovered that tau is highly decorated with post-translational modifications (PTMs) which dictate 1) tau's ability to associate with and enhance microtubule stability and 2) tau's proclivity towards oligomerization and aggregation, and thus often represent pathogenic tau strains. Several conditions and age-related diseases have been associated with misfolding and/or dysregulation of human tau protein such as, for example, Alzheimer's disease, Parkinson's disease, Parkinson's disease with dementia, dementia with Lewy bodies, amyotrophic lateral sclerosis, Down's syndrome, corticobasal degeneration, frontal temporal dementia, Pick's disease, multisystem atrophy, progressive supranuclear palsy, inclusion body myositis, prion protein cerebral amyoid angiopathy, argyrophilic grain disease, tangle predominant dementia, chronic traumatic encephalopathy, and traumatic brain injury.

There is a need in the art for effective compositions to detect, diagnose, and/or treat tauopathies and disorders and conditions associated with acetylated tau.

### SUMMARY OF THE INVENTION

The present invention is based on the identification of two acetylation sites within the human tau protein, K280 and K311, that are indicative of a disease state and, as such, represent diagnostic and/or therapeutic targets. Accordingly, one aspect of the invention relates to a monoclonal antibody or an antigen binding fragment thereof that specifically binds acetylated lysine 280 or acetylated lysine 311 in human tau protein.

Another aspect of the invention relates to a composition, e.g., a pharmaceutical composition, comprising one or more of the antibodies of the invention. In one embodiment, the composition comprises multiple antibodies to target both K280 and K311 simultaneously.

An additional aspect of the invention relates to a method of treating, preventing, or delaying progression of tauopathies and/or relates to preventing or delaying the normal aging process in a subject in need thereof, wherein the tauopathy comprises acetylation of a human Tau protein at K280 and/or K311, the method comprising delivering to the subject the antibodies of the invention, thereby treating, preventing, or delaying progression of the tauopathy and/or relates to preventing or delaying the normal aging process in the subject.

Another aspect of the invention relates to the use of the antibodies of the invention to treat, prevent, or delay progression of a disease or condition associated with a tau protein acetylated at lysine 280 and/or lysine 311, such as Alzheimer's disease, Parkinson's disease, amyotrophic lateral sclerosis, Down's syndrome, corticobasal degeneration, frontal temporal dementia, Pick's disease, Parkinson's disease with dementia, dementia with Lewy bodies, multisystem atrophy, progressive supranuclear palsy, inclusion body myositis, prion protein cerebral amyoid angiopathy, argyrophilic grain disease, tangle predominant dementia, chronic traumatic encephalopathy, and traumatic brain injury and/or prevent or delay progression of the normal aging process.

Another aspect of the invention relates to the antibody or antigen binding fragment thereof to human acetylated tau at K280 and/or K311 for use as a medicament, for use in diagnostics, and/or for detection purposes.

These and other aspects of the invention are set forth in more detail in the description of the invention below.

### BRIEF DESCRIPTION OF THE DRAWINGS

FIGS. 1A-ID are western blots of purified Ac-K280) antibodies in HEK293 cells with and without the acetylase transferase CREB binding protein (CBP/p300). FIGS. 1A-1C show the monoclonal antibodies bind preferentially to wild-type (WT) K280) compared to K280R mutants. Total protein western blots are shown as controls. FIG. 1D shows a western blot of cell extracts probed with total Ac-lysine (Cell signaling 9441) antibodies in HEK293 cells with and without the acetyl transferase CREB binding protein (CBP/p300). This shows further validation that Ac-K280 monoclonal antibodies bind preferentially to acetylated wild-type (WT) K280) compared to other acetylated lysine residues in HEK293 cells.

FIGS. 2A-2D are western blots of purified Ac-K311 antibodies in HEK293 cells with and without the acetylase transferase CREB binding protein (CBP/p300). FIGS. 2A-2C show the monoclonal antibodies bind preferentially to wild-type (WT) K311 compared to K311R mutants. Total protein western blots are shown as controls. FIG. 2D shows a western blot of cell extracts probed with total Ac-lysine (Cell signaling 9441) antibodies in HEK293 cells with and without the acetyl transferase CREB binding protein (CBP/p300). This shows further validation that Ac-K311 monoclonal antibodies bind preferentially to acetylated wild-type (WT) K311 compared to other acetylated lysine residues in HEK293 cells.

FIGS. 3A-3C are a series of immunocytochemistry images of Ac-K280 antibodies in primary cortical neurons. FIGS. 3A-3C show images with pUltra empty vector as a control, with WT tau, and with WT tau and the catalytic domain of CREB binding protein with a nuclear exclusion sequence. Monoclonal Ac-K280 antibodies are labeled with GFP and polyclonal Ac-K280 is labeled in RFP.

FIGS. 4A-4B are a series of immunocytochemistry images of Ac-K311 antibodies in primary cortical neurons. FIGS. 4A-4B show images with pUltra empty vector as a control, with WT tau, and with WT tau and the catalytic domain of CREB binding protein with a nuclear exclusion sequence. Monoclonal Ac-K311 antibodies are labeled with GFP and polyclonal Ac-K311 is labeled in RFP.

FIGS. 5A-5C are immunocytochemistry images of monoclonal antibodies delivered with carrier molecule in HEK293 cells. FIGS. 5A-5C show that the monoclonal antibodies 11D5 (Ac-K311) and 23B7 (Ac-K280) colocalize with EEA1 and Rab5 (endosomal), and LAMP1 (lysosomal) indicating the internalization of the antibodies.

FIG. 6 is a series of immunocytochemistry images of monoclonal antibodies in primary cortical neurons. FIG. 6 shows that the monoclonal antibodies are taken up by the primary cortical neurons and colocalize with Ac-tau varicosities induced by transduction with WT tau and the catalytic domain of CREB binding protein with a nuclear exclusion sequence.

FIG. 7 is a series immunohistochemistry images in Alzheimer disease post-mortem brain. FIG. 7 shows that the Ac-K280 monoclonal antibodies are able to detect amyloid plaques and neurofibrillary tangles in Alzheimer disease post-mortem brains.

FIGS. 8A-8B are a series of immmunohistochemistry images of mouse brain regions. FIG. 8A shows that the exemplary Ac-K280 clone 19B6 antibody detects tau pathology in PS19/5×FADmice. FIG. 8B shows Ac-K280 tau is present in aged PS19/5×FAD mice using Ac-K280 (polyclonal).

FIG. 9 provides immunochemistry images showing Ac-K311 tau is present in aged PS19/5×FAD mice. *=mice were sacrificed because they had reach end-point pathology.

FIG. 10 includes a series of hippocampal (Hp) and cortical (Ctx) images showing phosphorylated tau is present in aged PS19/5×FAD mice. *=mice were sacrificed because they had reach end-point pathology.

FIG. 11 is a series of hippocampal (Hp) and cortical (Ctx) images showing conformational tau (MC1) is present in aged PS19/5×FAD mice. *=mice were sacrificed because they had reach end-point pathology.

FIG. 12 is a series of fluorescent immunohistochemistry images of the hippocampus of aged mice using Ac-K280 (polyclonal) and 6E10 (amyloid beta) showing Ac-K280 and amyloid beta plaques accumulate in aged PS19/5×FAD mice. *=mice were sacrificed because they had reach end-point pathology.

FIG. 13 is an ELISA assay showing clones 19B6 and 17G8 preferentially detect acetylated vs. non-acetylated tau peptides. Two K280 clones (17G8 and 19B6) are shown.

FIGS. 14A-14B are images of human, Braak VI Alzheimer's disease post-mortem brain stained with monoclonal Ac-tau antibodies. Cortical brain sections (across three different cortical regions) from four separate AD patients were stained with both sets of (FIG. 14A) Ac-K280) and (FIG. 14B) Ac-K311 clones. White square represents magnified inset. Additional image of neurofibrillary tangle closely associated with amyloid beta plaques are included at the bottom (black border).

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See. e.g., Green et al., Molecular Cloning: A Laboratory Manual 4th Ed. (Cold Spring Harbor, NY, 2012): Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

### Definitions

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide sequence of this invention, means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to inhibit expression of a target mRNA of at least about 50% or more as compared to the expression level of a polynucleotide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of cancer, reduction in tumor burden, prevention of further tumor growth, prevention of metastasis, or increase in survival time). Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Prevent” or “preventing” or “prevention” refer to prevention or delay of the onset of the disorder and/or a decrease in the severity of the disorder in a subject relative to the severity that would develop in the absence of the methods of the invention. The prevention can be complete, e.g., the total absence of cancer in a subject. The prevention can also be partial, such that the occurrence or severity of cancer in a subject is less than that which would have occurred without the present invention.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

The term “antibody fragment” refers to a portion of an immunoglobulin, often the hypervariable region and portions of the surrounding heavy and light chains that displays specific binding affinity for a particular target, typically a molecule. A hypervariable region is a portion of an immunoglobulin that physically binds to the polypeptide target. An antibody fragment thus includes or consists of one or more portions of a full-length immunoglobulin retaining the targeting specificity of the immunoglobulin. Such antibody fragment may for instance lack at least partially the constant region (Fc region) of the full-length immunoglobulin. In some embodiments, an antibody fragment is produced by digestion of the full-length immunoglobulin. An antibody fragment may also be a synthetic or recombinant construct that contains one or more parts of the immunoglobulin or immunoglobulin chains (see e.g. HOLLIGER, P. and Hudson, J. Engineered antibody fragments and the rise of single domains. Nature Biotechnology 2005, vol. 23, no. 9, p. 1126-1136). Examples of an antibody fragment include, but are not limited to, an scFv, a Fab, a Fv, a Fab′, a F(ab′)₂ fragment, a dAb, a VHH, a nanobody, a V (NAR) or a so called minimal recognition unit.

A “single chain variable fragment” or a “single chain antibody” or an “scFv” are examples of a type of antibody fragment. An scFv is a fusion protein that includes the VH and VL domains of an immunoglobulin connected by a linker. It thus lacks the constant Fc region present in a full-length immunoglobulin.

A “monoclonal antibody or an antigen binding fragment thereof” as used herein refers to a full-length immunoglobulin, an antibody fragment, a proteinaceous non-immunoglobulin scaffold, and/or other binding compound, which has an immunoglobulin-like function. Typically the monoclonal antibody or an antigen binding fragment thereof is a proteinaceous binding molecule. Such monoclonal antibody or an antigen binding fragment thereof can be monovalent or multivalent, i.e. having one or more antigen binding sites. Non-limiting examples of monovalent binding members include scFv, Fab fragments, dAb, VHH, DARPins, affilins and nanobodies. A multivalent monoclonal antibody or an antigen binding fragment thereof can have two, three, four or more antigen binding sites whereby one or more different antigens can be recognized. Full length immunoglobulins, F(ab′)₂ fragments, bis-scFv (or tandem scFv) and diabodies are nonlimiting examples of multivalent monoclonal antibody or an antigen binding fragment thereof: in the exemplary multivalent monoclonal antibody or an antigen binding fragment thereof, two binding sites are present, i.e. the monoclonal antibody or an antigen binding fragment thereof is bivalent. In some embodiments, the multivalent monoclonal antibody or an antigen binding fragment thereof is bispecific, i.e. the monoclonal antibody or an antigen binding fragment thereof is directed against two different targets or two different target sites on one target molecule. Bispecific antibodies are, e.g., reviewed in MULLER, D. and Kontermann, R. E. Bispecific antibodies. Edited by DUBEL, S. Weinheim: Wiley-VCH, 2007. ISBN 3527314539. p. 345-378. In some embodiments, the multivalent monoclonal antibody or an antigen binding fragment thereof includes more than two, e.g., three or four different binding sites for three or four, respectively, different antigens. Such monoclonal antibody or an antigen binding fragment thereof is multivalent and multispecific, in particular tn- or tetra-specific, respectively.

“Non-antibody scaffolds” are antigen-binding polypeptides which are e.g. described in FIELDER, M. and Skerra, A. Non-antibody scaffolds. Edited by DUBEL, S. Weinheim: Wiley-VCH, 2007. ISBN 3527314539. p. 467-500; or GILBRETH, R. N. and Koide, S. Structural insights for engineering binding proteins based on non-antibody scaffolds. Curr Opin Struct Biol 2012, vol. 22, p. 4 13-420. Non-limiting examples include affibodies, affilin molecules, an AdNectin, a mutein based on a polypeptide of the lipocalin family (Anticalin®), a DARPin, Knottin, a Kunitz-type domain, an Avimer, a Tetranectin and a trans-body. Avimers contain so called A-domains that occur as strings of multiple domains in several cell surface receptors (Silverman, J., et al., Nature Biotechnology (2005) 23, 1556-1561). Tetranectins, derived from the respective human homotrimeric protein, likewise contain ioop regions in a C-type lectin domain that can be engineered for desired binding (ibid.).

Chemical and/or biological modifications may be conducted to optimize pharmacodynamics or water solubility of the protein or to lower its side effects. For example, PEGylation, PASylation and/or HESylation may be applied to slow down renal clearance and thereby increase plasma half-life time of the monoclonal antibody or an antigen binding fragment thereof. Additionally or alternatively, a modification may add a different functionality to the protein, e.g. a toxin to more efficiently combat cancer cells, or a detection molecule for diagnostic purposes.

Glycosylation refers to a process that attaches carbohydrates to proteins. In biological systems, this process is performed enzymatically within the cell as a form of co-translational and/or post-translational modification. A protein, here the monoclonal antibody or an antigen binding fragment thereof such as an antibody, can also be chemically glycosylated. Typically, but not limited to, glycosylation is (i)N-linked to a nitrogen of asparagine or arginine side-chains: (ii)O-linked to the hydroxy oxygen of serine, threonine, tyrosine, hydroxylysine, or hydroxyproline side-chains: (iii) involves the attachment of xylose, fucose, mannose, and N-acetylglucosamine to a phospho-serine: or (iv) in form of C-mannosylation wherein a mannose sugar is added to a tryptophan residue found in a specific recognition sequence. Glycosylation patterns can, e.g., be controlled by choosing appropriate cell lines, culturing media, protein engineering manufacturing modes and process strategies (HOSSLER, P. Optimal and consistent protein glycosylation in mammalian cell culture. Glycobiology 2009, vol. 19, no. 9, p. 936-949.).

Protein engineering to control or alter the glycosylation pattern may involve the deletion and/or the addition of one or more glycosylation sites. The creation of glycosylation sites can conveniently be accomplished by introducing the corresponding enzymatic recognition sequence into the amino acid sequence of the monoclonal antibody or an antigen binding fragment thereof or by adding or substituting one or more of the above enumerated amino acid residues.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and/or adenovirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992): Wu et al., J. Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987): Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, etc.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the term “over-expression” or “over-expressing” refers to increased levels of a polypeptide being produced and/or increased time of expression (e.g., constitutively expressed) compared to a wild-type cell.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary.” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least about 50%, 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

As used herein, the terms “contacting,” “introducing” and “administering” are used interchangeably, and refer to a process by which monoclonal antibodies or an antigen binding fragment thereof of the present invention or a nucleic acid molecule encoding a monoclonal antibodies or an antigen binding fragment thereof of this invention is delivered to a cell, in order to inhibit or alter or modify the presence and/or activity of a target protein (e.g. a misfolded and/or dysregulated protein especially through post-translational modifications). The monoclonal antibodies or an antigen binding fragment thereof may be administered in a number of ways, including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and mitochondrial genome, and therefore includes integration of the nucleic acid into, for example, the mitochondrial genome. Stable transformation as used herein can also refer to a transgene that is maintained extrachromasomally, for example, as a minichromosome.

Transient transformation may be detected by, for example, an enzyme-linked immunosorbent assay (ELISA) or western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism. Stable transformation of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Embodiments of the invention are directed to expression cassettes designed to express the nucleic acids of the present invention. As used herein, “expression cassette” means a nucleic acid molecule having at least a control sequence operably linked to a nucleotide sequence of interest. In this manner, for example, promoters in operable interaction with the nucleotide sequences for the monoclonal antibodies or an antigen binding fragment thereof of the invention are provided in expression cassettes for expression in an organism or cell.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences.

Furthermore, a “promoter” of this invention is a promoter capable of initiating transcription in a cell of an organism. Such promoters include those that drive expression of a nucleotide sequence constitutively, those that drive expression when induced, and those that drive expression in a tissue- or developmentally-specific manner, as these various types of promoters are known in the art.

For purposes of the invention, the regulatory regions (i.e., promoters, transcriptional regulatory regions, and translational termination regions) can be native/analogous to the organism or cell and/or the regulatory regions can be native/analogous to the other regulatory regions. Alternatively, the regulatory regions may be heterologous to the organism or cell and/or to each other (i.e., the regulatory regions). Thus, for example, a promoter can be heterologous when it is operably linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and polynucleotide) are substantially modified from their original form and/or genomic locus, or the promoter is not the native promoter for the operably linked polynucleotide.

The choice of promoters to be used depends upon several factors, including, but not limited to, cell- or tissue-specific expression, desired expression level, efficiency, inducibility and selectability. For example, where expression in a specific tissue or organ is desired, a tissue-specific promoter can be used, such as camkII and synapsin for delivery to neurons. In contrast, where expression in response to a stimulus is desired, an inducible promoter can be used. Where continuous expression is desired throughout the cells of an organism, a constitutive promoter can be used. It is a routine matter for one of skill in the art to modulate the expression of a nucleotide sequence by appropriately selecting and positioning promoters and other regulatory regions relative to that sequence.

In addition to the promoters described above, the expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

The expression cassette also can optionally include a transcriptional and/or translational termination region (i.e., termination region) that is functional in the organism. A variety of transcriptional terminators are available for use in expression cassettes and are responsible for the termination of transcription beyond the transgene and correct mRNA polyadenylation. The termination region may be native to the transcriptional initiation region, may be native to the operably linked nucleotide sequence of interest, may be native to the host, or may be derived from another source (i.e., foreign or heterologous to the promoter, the nucleotide sequence of interest, the host, or any combination thereof).

A signal sequence can be operably linked to nucleic acids of the present invention to direct the nucleotide sequence into a cellular compartment or to be secreted from the cell. In this manner, the expression cassette will comprise a nucleotide sequence encoding the monoclonal antibodies or an antigen binding fragment thereof operably linked to a nucleic acid sequence for the signal sequence. The signal sequence may be operably linked at the N- or C-terminus of the monoclonal antibodies or an antigen binding fragment thereof.

Regardless of the type of regulatory sequence(s) used, they can be operably linked to the nucleotide sequence of the monoclonal antibodies or an antigen binding fragment thereof. As used herein, “operably linked” means that elements of a nucleic acid construct such as an expression cassette are configured so as to perform their usual function. Thus, regulatory or control sequences (e.g., promoters) operably linked to a nucleotide sequence of interest are capable of effecting expression of the nucleotide sequence of interest. The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence. A nucleotide sequence of the present invention (i.e., encoding a monoclonal antibodies or an antigen binding fragment thereof) can be operably linked to a regulatory sequence, thereby allowing its expression in a cell and/or subject.

The expression cassette also can include a nucleotide sequence for a selectable marker, which can be used to select a transformed organism or cell. As used herein, “selectable marker” means a nucleic acid that when expressed imparts a distinct phenotype to the organism or cell expressing the marker and thus allows such transformed organisms or cells to be distinguished from those that do not have the marker. Such a nucleic acid may encode either a selectable or screenable marker, depending on whether the marker confers a trait that can be selected for by chemical means, such as by using a selective agent (e.g., an antibiotic or the like), or on whether the marker is simply a trait that one can identify through observation or testing, such as by screening (. Of course, many examples of suitable selectable markers are known in the art and can be used in the expression cassettes described herein.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988): Biocomputing: Informatics and Genome Projects (Smith, D. W., ed.) Academic Press, New York (1993): Computer Analysis of Sequence Data. Part I (Griffin, A. M., and Griffin, H. G., eds.) Humana Press, New Jersey (1994): Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10: Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48: 1073 (1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894: see BLAST Manual, Altschul et al., NCBI, NLM, NIH: (Altschul et al., J. Mol. Biol. 215:403-410 (1990)): version 2.0 or higher of BLAST programs allows the introduction of gaps (deletions and insertions) into alignments: for peptide sequence BLASTX can be used to determine sequence identity; and, for polynucleotide sequence BLASTN can be used to determine sequence identity.

### Acetylated Tau

Tau phosphorylation has traditionally been viewed as the dominant modification controlling tau function in AD, and thus has been a primary target of tau immunotherapies. However, tau can also be acetylated as a post-translational modification, which regulates both normal tau function as well as abnormal pathogenic features, possessing gain-of-function and loss-of-function properties. Unlike phosphorylation, tau acetylation can occur primarily in the microtubule binding region (MTBR)—a region responsible for tau binding to MT. Tau acetylation can favor tau beta strand stacking, consistent with the role of acetylation in dictating the core tau protofilament discovered by cryo-EM. Indeed, tau acetylation can inhibit chaperone mediated autophagy (Caballero et al., Nat Commun. 2021 Apr. 14: 12 (1): 2238: doi: 10.1038/s41467-021-22501-9), promote aberrant tau aggregation, proteolytic cleavage, and impair microtubule (MT) binding. Furthermore, in PS19 mice, acetylated tau (Ac-tau) accumulation and AD pathology can be exacerbated by loss of HDAC6, a tau deacetylase. In human AD brain, tau acetylation, at a specific key residue (K280, residue numbering based on the wild-type human tau sequence (SEQ ID NO:1)) within the tau MTBR, can be detected within tau tangles, but in no circumstances is this modification detected in control, non-tauopathy individuals. In addition to residue K280, prominent acetylation can be detected at the homologous tau residue in the third MTBR repeat (K311). Acetylation can be identified on transmissible tau seeds leading to their ability to corrupt normal tau. Interestingly, acetylated tau can also be a pathological marker of brain injury, and reducing acetylated tau levels can rescue cognitive decline in a mouse model of brain injury and neurodegeneration, indicating acetylated tau directed immunotherapies may also be efficacious in tramatic brain injury (TBI) and other non-AD tauopathies and may be efficacious in preventing and/or delaying onset of normal age progression.

Wild-type human tau sequence (Tau-441, (2N4R) isoform) (SEQ ID NO:1):

MAEPRQEFEV MEDHAGTYGL GDRKDQGGYT MHQDQEGDTD
AGLKESPLQT PTEDGSEEPG SETSDAKSTP TAEDVTAPLV
DEGAPGKQAA AQPHTEIPEG TTAEEAGIGD TPSLEDEAAG
HVTQARMVSK SKDGTGSDDK KAKGADGKTK IATPRGAAPP
GQKGQANATR IPAKTPPAPK TPPSSGEPPK SGDRSGYSSP
GSPGTPGSRS RTPSLPTPPT REPKKVAVVR TPPKSPSSAK
SRLQTAPVPM PDLKNVKSKI GSTENLKHQP GGGKVQIINK
KLDLSNVQSK CGSKDNIKHV PGGGSVQIVY KPVDLSKVTS
KCGSLGNIHH KPGGGQVEVK SEKLDFKDRV QSKIGSLDNI
THVPGGGNKK IETHKLTFRE NAKAKTDHGA EIVYKSPVVS
GDTSPRHLSN VSSTGSIDMV DSPQLATLAD EVSASLAKQG L

Intriguingly, the K311 and K280 residues are located at homologous sites of tau, VQIVYK (SEQ ID NO:2) and VQIINK (SEQ ID NO:3), respectively, which are responsible for tau's ability to self-aggregate. Given the critical role of these two particular acetylated residues in promoting tau aggregation and preventing MT binding, these complimentary acetylated epitopes are attractive targets for immunotherapy. Using an immunotherapy against Ac-tau possesses several advantages over targeting Ac-tau via pharmacological inhibitors-in particular, immunotherapies offer a higher degree of specificity and avoid potential off-target effects. Several pre-clinical and clinical trials (currently 11) targeting total tau and specific, pathogenic strains of tau are ongoing. Pre-clinical immunotherapy trials have found varying levels of success in alleviating tau pathology (hyperphosphorylation, insolubility, tau seeding and spread) and associated behavioral abnormalities (motor, memory). Five clinical trials have found evidence of target engagement and have progressed towards Phase 2 efficacy trials.

### Antibodies

The inventors have identified and characterized antibodies that specifically bind to human tau acetylated at the K280 and/or the K311 positions. Not wanting to be limited by theory, it is believed that this antibody bound to acetylated tau facilitates subsequent depletion of acetylated tau. Antibody-based immunotherapies are primarily thought to clear proteins by 1) binding proteins extracellularly, preventing cellular uptake and prion-like transfer of tau and/or 2) entering cells via receptor mediated endocytosis and binding proteins intracellularly to target them for degradation by endosomal autophagy-lysosomal pathways. Such antibodies can advantageously be used to deplete acetylated tau in a subject, e.g., for research or therapeutic purposes. Such antibodies can be used to treat disorders and/or the normal aging process associated with acetylated tau. Accordingly, one aspect of the invention relates to antibodies or fragments thereof that specifically bind to acetylated tau at the K280 and/or the K311 positions and deplete acetylated tau when administered to a subject. The term “antibody” or “antibodies” as used herein refers to all types of

immunoglobulins, including IgG, IgM, IgA, IgD, and IgE. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, Fab′, F(ab′)₂, and Fv fragments; domain antibodies, diabodies: vaccibodies, linear antibodies: single-chain antibody molecules; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be con structed to allow rapid and easy identification of monoclonal Fab fragments with the desired specificity (Huse et al., Science 254:1275 (1989)).

A monoclonal antibody or an antigen binding fragment thereof as disclosed herein may be PEGylated, or glycosylated or Hesylated if desired. In some embodiments a monoclonal antibody or an antigen binding fragment thereof is a fusion protein of one of the exemplary proteinaceous binding molecules above and an albumin-binding domain, for instance an albumin-binding domain of streptococcal protein G. In some embodiments a monoclonal antibody or an antigen binding fragment thereof is a fusion protein of an immunoglobulin fragment, such as a single-chain diabody, and an immunoglobulin binding domain, for instance a bacterial immunoglobulin binding domain. As an illustrative example, a single-chain diabody may be fused to domain B of staphylococcal protein A as described by Unverdorben et al. (Protein Engineering, Design & Selection 25, 8 1-88).

Antibodies of the invention may be altered or mutated for compatibility with species other than the species in which the antibody was produced. For example, antibodies may be humanized or camelized. Humanized forms of nonhuman (e.g., murine) antibodies are chimeric immunoglobulins, immunoglobulin chains or fragments thereof (such as Fv. Fab. Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a nonhuman species (donor antibody) such as mouse, rat, or rabbit having the desired specificity, affinity, and capacity. In some instances. Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also will comprise at least a portion of an immunoglobulin constant region (Fc), typically that of a human immunoglobulin (Jones et al., Nature 321: 522 (1986); Riechmann et al., Nature. 332:323 (1988); and Presta. Curr Op. Struct. Biol. 2:593 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can essentially be performed following the method of Winter and co-workers (Jones et al., Nature 321:522 (1986): Riechmann et al., Nature 332:323 (1988): Verhoeven et al., Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues (e.g., all of the CDRs or a portion thereof) and possibly some FR residues are substituted by residues from analogous sites in rodent antibodies.

In one embodiment, the monoclonal antibody is humanized. In one embodiment the monoclonal antibody is chimeric. In one embodiment the monoclonal antibody is an antibody fragment such as a Fab, a Fab′, a F(ab)′₂, a scFv, a Fv fragment, a nanobody, an intrabody, a VHH, or a minimal recognition unit and/or a full-length immunoglobulin molecule and/or a non-immunoglobulin scaffold such as an affibody, an affilin molecule, an AdNectin, a lipocalin mutein, a DARPin, a Knottin, a Kunitz-type domain, an Avimer, a Tetranectin, or a trans-body. In one embodiment, the monoclonal antibody is monovalent or multivalent, and optionally bispecific, preferably a diabody, a single-chain diabody, or a tandem scFv.

Human antibodies can also be produced using various techniques known in the art, including phage display libraries (Hoogenboom and Winter, J. Mol. Biol. 227:381 (1991): Marks et al., J. Mol. Biol. 222:581 (1991)). The techniques of Cole et al, and Boemer et al. are also available for the preparation of human monoclonal antibodies (Cole et al., Monoclonal Antibodies and Cancer Therapy, Alan R. Liss, p. 77 (1985) and Boemer et al., J. Immunol. 147:86 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807:5,545,806; 5,569,825:5,625,126:5,633,425:5, 661,016, and in the following scientific publications: Marks et al., Bio/Technology 10:779 (1992): Lonberg et al., Nature 368:856 (1994): Morrison, Nature 368:812 (1994); Fishwild et al., Nature Biotechnol. 14:845 (1996): Neuberger, Nature Biotechnol. 14:826 (1996): Lonberg and Huszar, Intern. Rev. Immunol. 13:65 (1995).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures. The polynucleotide sequence and polypeptide sequence of tau is known in the art and can be found in sequence databases such as GenBank. Examples of sequences include the human tau polypeptide sequence (Accession No. NP_005901) and polynucleotide sequence (Accession No. NM_005910), incorporated herein by reference in their entirety.

Monoclonal antibodies used to carry out the present invention can be produced in a hybridoma cell line according to the technique of Kohler and Milstein, Nature 265:495 (1975). For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., Huse, Science 246:1275 (1989).

Antibodies specific to the target polypeptide can also be obtained by phage display techniques known in the art.

Various immunoassays can be used for screening to identify antibodies having the desired specificity for acetylated tau. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). A two-site, monoclonal-based immunoassay utilizing monoclonal antibodies reactive to two non-interfering epitopes on the polypeptides or peptides of this invention can be used as well as a competitive binding assay.

Antibodies can be conjugated to a solid support (e.g., beads, plates, slides or wells formed from materials such as latex or polystyrene) in accordance with known techniques. Antibodies can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Determination of the formation of an antibody/antigen complex in the methods of this invention can be by detection of, for example, precipitation, agglutination, flocculation, radioactivity, color development or change, fluorescence, luminescence, etc., as is well known in the art.

In one embodiment, the antibody is an antibody or a fragment thereof (e.g., a monoclonal antibody or a fragment thereof) that specifically binds to acetylated tau at K280 or K311. The antibody may bind to one of these specific epitopes on tau.

In one embodiment, the antibody is a monoclonal antibody or a fragment thereof produced by one of the hybridoma cell lines listed in Table 1. In a further embodiment, the antibody is a monoclonal antibody or a fragment thereof that competes for binding to the same epitope specifically bound by the monoclonal antibody produced by the hybridoma cell line. In another embodiment, the antibody is a monoclonal antibody or a fragment thereof that specifically binds to the same epitope specifically bound by the monoclonal antibody produced by hybridoma cell line. In certain embodiments, the monoclonal antibody or a fragment thereof is a chimeric antibody or a humanized antibody. In additional embodiments, the chimeric or humanized antibody comprises at least a portion of the CDRs of the monoclonal antibody produced by the hybridoma cell line. As used herein, a “portion” of a CDR is defined as one or more of the three loops from each of the light and heavy chain that make up the CDRs (e.g., from 1-6 of the CDRs) or one or more portions of a loop comprising, consisting essentially of, or consisting of at least three contiguous amino acids. For example, the chimeric or humanized antibody may comprise 1, 2, 3, 4, 5, or 6 CDR loops, portions of 1, 2, 3, 4, 5, or 6 CDR loops, or a mixture thereof, in any combination.

In an embodiment the monoclonal antibody, or an antigen binding fragment thereof, is specific for Ac-K280 and may comprise a CDR1, a CDR2, and/or a CD3 sequence from Table 2A or Table 2B. In an embodiment, the monoclonal antibody, or an antigen binding fragment thereof, specific for Ac-K280) comprises, consists essentially of, and/or consists of an amino acid sequence of contiguous amino acids identical or almost identical (e.g., 90%, 92%, 95%, 98%, 99% identical) to a heavy-chain CDR selected from Table 2A. In an embodiment, the monoclonal antibody, or an antigen binding fragment thereof, specific for Ac-K280 comprises a light-chain CDR selected from Table 2B. In an example embodiment, the monoclonal antibody, or an antigen binding fragment thereof, specific for Ac-K280) comprises, consists essentially of, and/or consists of an amino acid sequence of contiguous amino acids identical or almost identical (e.g., 90%, 92%, 95%, 98%, 99% identical) to a sequence selected from SEQ ID NOs: 18-43.

In an embodiment the monoclonal antibody, or an antigen binding fragment thereof, is specific for Ac-K311 and may comprise a CDR1, a CDR2, and/or a CDR3 sequence from Table 3A or Table 3B. In an embodiment, the monoclonal antibody, or an antigen binding fragment thereof, specific for Ac-K311 comprises, consists essentially of, and/or consists of an amino acid sequence of contiguous amino acids identical or almost identical (e.g., 90%, 92%, 95%, 98%, 99% identical) to a heavy-chain CDR selected from Table 3A. In an embodiment, the monoclonal antibody, or an antigen binding fragment thereof, specific for Ac-K311 comprises, consists essentially of, and/or consists of an amino acid sequence of contiguous amino acids identical or almost identical (e.g., 90%, 92%, 95%, 98%, 99% identical) to a light-chain CDR selected from Table 3B. In an example embodiment, the monoclonal antibody specific for Ac-K311, or an antigen binding fragment thereof, comprises, consists essentially of, and/or consists of an amino acid sequence of contiguous amino acids identical or almost identical (e.g., 90%, 92%, 95%, 98%, 99% identical) to a sequence selected from SEQ ID NOs: 44-65.

In an aspect, a monoclonal antibody specific for Ac-K280, or an antigen binding fragment thereof, may comprise a K280 monoclonal heavy chain CDR1 sequence that comprises, consists essentially of, and/or consists of an amino acid sequence of contiguous amino acids identical or almost identical (e.g., 90%, 92%, 95%, 98%, 99% identical) to a sequence represented by the formula GX₁NIX₂DX₃X₄ (SEQ ID NO:4), wherein X₁ is L or F, X₂ is K or E, X₃ is D or Y, and X₄ is Y or F. In an aspect, a monoclonal antibody specific for Ac-K280, or an antigen binding fragment thereof, may comprise a K280 monoclonal heavy chain CDR2 sequence that comprises, consists essentially of, and/or consists of an amino acid sequence of contiguous amino acids identical or almost identical (e.g., 90%, 92%, 95%, 98%, 99% identical) to a sequence represented by the formula IDPEX₁X₂X₃X₄ (SEQ ID NO:5), wherein X₁ is N or D, X₂ is G or D, X₃ is D, E, or K, and X₄ is P. T, or N. In an aspect, a monoclonal antibody specific for Ac-K280, or an antigen binding fragment thereof, may comprise a K280 monoclonal heavy chain CDR3 sequence that comprises, consists essentially of, and/or consists of an amino acid sequence of contiguous amino acids identical or almost identical (e.g., 90%, 92%, 95%, 98%, 99% identical) to a sequence represented by the formula X₁TRGX₂, wherein X₁ is A or T, and X₂ is L or V: the K280 monoclonal heavy chain CDR3 in other embodiments may comprise, consist essentially of, and/or consist of an amino acid sequence of contiguous amino acids identical or almost identical (e.g., 90%, 92%, 95%, 98%, 99% identical) to a sequence represented by the formula X₁IRDFEV (SEQ ID NO: 6), wherein X₁ is F or no amino acid. In an embodiment, the K280 monoclonal antibody may comprise, consist essentially of, and/or consist of an amino acid sequence comprising one or more of the CDR1, CDR2, CDR3 heavy chain sequences according to the preceding formulae.

In an embodiment, a monoclonal antibody specific for Ac-K280, or an antigen binding fragment thereof, may comprise a K280 monoclonal light chain CDR1 sequence that comprises, consists essentially of, and/or consists of an amino acid sequence of contiguous amino acids identical or almost identical (e.g., 90%, 92%, 95%, 98%, 99% identical) to a sequence represented by the formula SSX₁SSX₂X₃ (SEQ ID NO:7), wherein X₁ is V or I, X₂ is S or T, and X₃ is Y or F. In one embodiment, a monoclonal antibody specific for Ac-K280, or an antigen binding fragment thereof, may comprise a K280) monoclonal light chain CDR1 sequence that comprises, consists essentially of, and/or consists of an amino acid sequence of contiguous amino acids identical or almost identical (e.g., 90%, 92%, 95%, 98%, 99% identical) to a sequence represented by the formula QX₁IVHX₂X₃GHTY (SEQ ID NO:8), wherein X₁ is S or N, X₂ is G or D, and X₃ is S or N. In an aspect, a monoclonal antibody specific for Ac-K280, or an antigen binding fragment thereof, may comprise a K280 monoclonal light chain CDR2 sequence that comprises, consists essentially of, and/or consists of an amino acid sequence of contiguous amino acids identical or almost identical (e.g., 90%, 92%, 95%, 98%, 99% identical) to a sequence represented by the formula X₁X₂S, wherein X₁ is S, K, or R, and X₂ is T or V. In an aspect, a monoclonal antibody specific for Ac-K280, or an antigen binding fragment thereof, may comprise a K280 monoclonal light chain CDR3 sequence that comprises, consists essentially of, and/or consists of an amino acid sequence of contiguous amino acids identical or almost identical (e.g., 90%, 92%, 95%, 98%, 99% identical) to a sequence represented by the formula X₁QX₂X₃X₄PLT (SEQ ID NO:9) wherein X₁ is H or F, X₂ is Y or G, X₃ is H, S, or R, and X₄ is S, V, or I. In an embodiment, the monoclonal antibody specific for Ac-K280, or an antigen binding fragment thereof, may comprise, consist essentially of, and/or consist of an amino acid sequence comprising one or more of CDR1, CDR2, CDR3 light chain sequences according to the preceding formulae.

In an aspect, a monoclonal antibody specific for Ac-K311, or an antigen binding fragment thereof, may comprise a K311 monoclonal heavy chain CDR1 sequence that comprises, consists essentially of, and/or consists of an amino acid sequence of contiguous amino acids identical or almost identical (e.g., 90%, 92%, 95%, 98%, 99% identical) to a sequence represented by the formula GX₁TX₂X₃X₄YG (SEQ ID NO: 10), wherein X₁ is Y or F. X₂ is F or G, X₃ is S or T, and X₄ is S, N or T. In an aspect, monoclonal antibody specific for Ac-K311, or an antigen binding fragment thereof, may comprise a K311 monoclonal heavy chain CDR2 sequence that comprises, consists essentially of, and/or consists of an amino acid sequence of contiguous amino acids identical or almost identical (e.g., 90%, 92%, 95%, 98%, 99% identical) to a sequence represented by the formula IX₁X₂GX₃X₄ X₅T, wherein X₁ is Y or S, X₂ is I, Y, or F, X₃ is N, S, or G, X₄ is T or G, and X₅ is Y or F. In an aspect, K311 monoclonal heavy chain CDR3 may comprise, consist essentially of, and/or consist of an amino acid sequence of contiguous amino acids identical or almost identical (e.g., 90%, 92%, 95%, 98%, 99% identical) to a sequence represented by the formula X₁RWX₂X₃GYX₄FDY (SEQ ID NO: 11), wherein X₁ is A, G or V, X₂ is R or V, X₃ is T, P, or N, and X₄ is F or Y. In an aspect, the K311 monoclonal heavy chain CDR3 may comprise, consist essentially of, and/or consist of an amino acid sequence of contiguous amino acids identical or almost identical (e.g., 90%, 92%, 95%, 98%, 99% identical) to a sequence according to SSLWETWLAY (SEQ ID NO:12). In an embodiment, the K311 monoclonal antibody may comprise, consist essentially of, and/or consist of an amino acid sequence comprising one or more of CDR1, CDR2, CDR3 heavy chain sequences according to the preceding formulae.

In an aspect, a monoclonal antibody specific for Ac-K311, or an antigen binding fragment thereof, may comprise a K311 monoclonal light chain CDR1 that comprises, consists essentially of, and/or consists of an amino acid sequence of contiguous amino acids identical or almost identical (e.g., 90%, 92%, 95%, 98%, 99% identical) to a sequence represented by the formula X₁X₂IX₃HX₄NGX₅TY (SEQ ID NO: 13), wherein X₁ is Q, R or L, X₂ is N, T or S, X₃ is V or I, X₄ is S or T, and X₅ is N or D. In an aspect, a monoclonal antibody specific for Ac-K311, or an antigen binding fragment thereof, may comprise a K311 monoclonal light chain CDR2 that comprises, consists essentially of, and/or consists of an amino acid sequence of contiguous amino acids identical or almost identical (e.g., 90%, 92%, 95%, 98%, 99% identical) to a sequence represented by the formula X₁VS, wherein X₁ is K or R. In an aspect, a monoclonal antibody specific for Ac-K311, or an antigen binding fragment thereof, may comprise a K311 monoclonal light chain CDR3 that comprises, consists essentially of, and/or consists of an amino acid sequence of contiguous amino acids identical or almost identical (e.g., 90%, 92%, 95%, 98%, 99% identical) to a sequence represented by the formula FQX₁SX₂X₃PX₄T (SEQ ID NO: 14), wherein X₁ is S, T, or G, X₂ is L or H, X₃ is V or I, and X₄ is L or P. In an embodiment, the monoclonal antibody specific for Ac-K311, or an antigen binding fragment thereof, may comprise, consist essentially of, and/or consist of an amino acid sequence comprising one or more of CDR1, CDR2, CDR3 light chain sequences according to the preceding formulae.

In an embodiment, the K311 monoclonal antibody may comprise, consist essentially of, and/or consist of an amino acid sequence comprising one or more of CDR1, CDR2, CDR3 heavy chain sequences, and/or one or more of CDR1, CDR2, and/or CDR3 light chain sequences disclosed herein.

It may be desirable to PEGylate the monoclonal antibody or an antigen binding fragment thereof. PEGylation may alter the pharmacodynamic and pharmacokinetic properties of a protein. Polyethylene-glycol (PEG) of an appropriate molecular weight is covalently attached to the protein backbone (see, e.g., PASUT, G, and Veronese, F. State of the art in PEGylation: the great versatility achieved after forty years of research. J Control Release 2012, vol. 161, no. 2, p. 461-472). PEGylation may additionally reduce the immunogenicity by shielding the PEGylated protein from the immune system and/or alter its pharmacokinetics by, e.g., increasing the in vivo stability of the monoclonal antibody or an antigen binding fragment thereof, protecting it from proteolytic degradation, extending its half-life time and by altering its biodistribution.

Similar effects may be achieved by PEG mimetics, e.g., HESylating or PASylating the antibody. HESylation utilizes hydroxyethyl starch (“HES”) derivatives, whereas during PASylation the antibody becomes linked to conformationally disordered polypeptide sequences composed of the amino acids proline, alanine and serine. These PEG mimetics and related compounds are, e.g., described in BINDER, U, and Skerra, A. Half-Life Extension of Therapeutic Proteins via Genetic Fusion to Recombinant PEG Mimetics, in Therapeutic Proteins: Strategies to Modulate Their Plasma Half-Lives. Edited by KONTERMANN, R., Weinheim, Germany: Wiley-VCH, 2012. ISBN: 9783527328499, p. 63-81.

In one embodiment, the monoclonal antibody is chemically or biologically modified, and optionally glycosylated, PEGylated, or HESylated.

The monoclonal antibody or an antigen binding fragment thereof may include an epitope such as a salvage receptor binding epitope. Such salvage receptor binding epitope typically refers to an epitope of the Fc region of an IgG molecule (e.g., IgG1, IgG2, IgG3, or IgG4) and has the effect of increasing the in vivo half-life of the molecule.

Additionally or alternatively, the monoclonal antibody or an antigen binding fragment thereof is labelled with or conjugated to a second moiety which ascribes ancillary functions following target binding. The second moiety may, e.g., have an additional immunological effector function, be effective in drug targeting or useful for detection, without being limited thereto. The second moiety can, e.g., be chemically linked or fused genetically to the monoclonal antibody or an antigen binding fragment thereof using known methods in the art.

Molecules which may serve as second moiety include, without being limited to, a radionuclide, also called a radioisotope, an apoenzyme, an enzyme, a co-factor, a peptide moiety such as a HIS-tag, a protein, a carbohydrate such as a mannose-6-phosphate tag, a fluorophore such as fluorescein isothiocyanate (FITC), phycoerythrin, a green/blue/red or other fluorescent protein, allophycocyanin (APC), a chromophore, a vitamin such as biotin, a chelator, an antimetabolite such as methotrexate, a liposome, a toxin such as a cytotoxic drug, or a radiotoxin. Illustrative examples of a radionuclide are ³⁵S, ³²P, ¹⁴C, ¹⁸F, and ¹²⁵I. Examples of suitable enzymes include, but are not limited to, alkaline phosphatase, horseradish peroxidase, beta-galactosidase and angiogenin. An illustrative example of a suitable protein is a lectin. Examples of suitable cytotoxic drugs include, but are not limited to, taxol, gramicidin D and colchicine.

A labelled monoclonal antibody or an antigen binding fragment thereof is particularly useful for in vitro and in vivo detection or diagnostic purposes. For example, a monoclonal antibody or an antigen binding fragment thereof labelled with a suitable radioisotope, enzyme, fluorophore or chromophore can be detected by radioimmunoassay (RIA), enzyme-linked immunosorbent assay (ELISA), or flow cytometry-based single cell analysis (e.g., FACS analysis), respectively. Similarly, the nucleic acids and/or vectors disclosed herein can be used for detection or diagnostic purposes, e.g., using labelled fragments thereof as probes in hybridization assays. Labelling protocols may, e.g., be found in JOHNSON, I, and Spence, M. T. Z. Molecular Probes Handbook, A Guide to Fluorescent Probes and Labeling Technologies. Life Technologies, 2010. ISBN: 0982927916.

It is to be understood that the outlined above also applies to T-bodies.

### Nucleic Acids

In one aspect, the invention relates to an isolated polynucleotide encoding a monoclonal antibody or an antigen binding fragment thereof to acetylated tau or a functional fragment thereof.

One embodiment of the invention is an isolated polynucleotide sequence encoding a monoclonal antibody or an antigen binding fragment thereof to acetylated tau or a functional fragment thereof, a plasmid or vector containing the isolated polynucleotide sequence, or a transfected cell containing the plasmid or vector or the isolated polynucleotide sequence. Another aspect of the invention relates to a vector comprising the polynucleotides or expression cassettes of the invention (e.g., encoding the humanized monoclonal antibody, the chimeric monoclonal antibody, a fragment such as a Fab, a Fab′, a F(ab)′₂, a scFv, a Fv fragment, a nanobody, an intrabody, a VHH, or a minimal recognition unit and/or a full-length immunoglobulin molecule and/or a non-immunoglobulin scaffold such as an affibody, an affilin molecule, an AdNectin, a lipocalin mutein, a DARPin, a Knottin, a Kunitz-type domain, an Avimer, a Tetranectin, or a trans-body, or an antibody that is monovalent or multivalent, and optionally bispecific, preferably a diabody, a single-chain diabody, or a tandem scFv). Suitable vectors include, but are not limited to, a plasmid, phage, viral vector (e.g., an AAV vector, an adenovirus vector, a herpesvirus vector, an alphavirus vector, or a baculovirus vector), bacterial artificial chromosome (BAC), or yeast artificial chromosome (YAC). For example, the nucleic acid can comprise, consist of, or consist essentially of an AAV vector comprising a 5′ and/or 3′ terminal repeat (e.g., 5′ and/or 3′ AAV terminal repeat). In some embodiments, the vector is a viral vector, e.g., a parvovirus vector, e.g., an AAV vector, e.g., an AAV8 or AAV9 vector. The viral vector may further comprise a nucleic acid comprising a recombinant viral template, wherein the nucleic acid is encapsidated by the parvovirus capsid. The invention further provides a recombinant parvovirus particle (e.g., a recombinant AAV particle) comprising the polynucleotides of the invention.

### Pharmaceutical Compositions

As a further aspect, the invention provides pharmaceutical formulations and methods of administering the same to achieve any of the therapeutic effects (e.g., treatment of tauopathy) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

One embodiment of the invention is a composition including an isolated polynucleotide sequence encoding a monoclonal antibody or an antigen binding fragment thereof to acetylated tau or a functional fragment thereof, a plasmid or vector containing the isolated polynucleotide sequence, or a transfected cell containing the plasmid or vector or the isolated polynucleotide sequence and a suitable carrier, diluent, or excipient, and optionally a pharmaceutically acceptable carrier, diluent, or excipient. In one embodiment, the composition is in a form suitable for parenteral, oral, rectal, systemic, urogenital, topical, intravitreal, intraocular, otic, intranasal, dermal, sublingual, or buccal administration.

The monoclonal antibody or an antigen binding fragment thereof of the invention can be formulated for administration in a pharmaceutical carrier in accordance with known techniques. See, e.g., Remington, The Science And Practice of Pharmacy (23rd Ed. 2020). In the manufacture of a pharmaceutical formulation according to the invention, the monoclonal antibody or an antigen binding fragment thereof (including the physiologically acceptable salts thereof) is typically admixed with, inter alia, an acceptable carrier. The carrier can be a solid or a liquid, or both, and is preferably formulated with monoclonal antibody or an antigen binding fragment thereof as a unit-dose formulation, for example, a tablet, which can contain from 0.01 or 0.5% to 95% or 99% by weight of the monoclonal antibody or an antigen binding fragment thereof. One or more monoclonal antibody or an antigen binding fragment thereof can be incorporated in the formulations of the invention, which can be prepared by any of the well-known techniques of pharmacy.

A further aspect of the invention is a method of treating subjects in vivo, comprising administering to a subject a pharmaceutical composition comprising a monoclonal antibody or an antigen binding fragment thereof of the invention in a pharmaceutically acceptable carrier, wherein the pharmaceutical composition is administered in a therapeutically effective amount. Administration of the monoclonal antibody or an antigen binding fragment thereof of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987): Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa). The therapeutically effective dosage of any specific compound will vary somewhat from compound to compound, and patient to patient, and will depend upon the condition of the patient and the route of delivery. As a general proposition, a dosage from about 0.001 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkey's, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of a tauopathy such as AD. In certain embodiments, the subject has or is at risk for a tauopathy such as AD.

### Methods

As one aspect, the invention provides methods of producing the monoclonal antibody or an antigen binding product thereof comprising cultivating a host cell comprising a nucleic acid encoding the monoclonal antibody or an antigen binding product thereof, thereby allowing the monoclonal antibody or an antigen binding fragment thereof to be expressed, recovering the monoclonal antibody or an antigen binding fragment thereof, and optionally purifying the monoclonal antibody or an antigen binding fragment thereof.

As one aspect, the invention provides methods of producing the monoclonal antibody or an antigen binding product thereof comprising contacting a cell-free expression system with a nucleic acid product template, the nucleic acid product template encoding the monoclonal antibody or an antigen binding fragment thereof, allowing transcription and translation of the nucleic acid product template to occur, thereby allowing a reaction mixture to be formed, recovering the monoclonal antibody or an antigen binding fragment thereof from the reaction mixture, and optionally purifying the monoclonal antibody or an antigen binding fragment thereof.

As one aspect, the invention provides methods of treating, preventing, or delaying progression of a disorder or condition associated with acetylated tau in a subject, comprising delivering to the subject a therapeutically effective amount of an antibody or a fragment thereof that specifically binds to acetylated K280 or acetylated K311, thereby treating the disorder or condition.

As one aspect, the invention provides methods of preventing or delaying progression of the normal aging process associated with acetylated tau in a subject, comprising delivering to the subject a therapeutically effective amount of an antibody or a fragment thereof that specifically binds to acetylated K280 or acetylated K311, thereby preventing or delaying progression of the normal aging process. Acetylation of soluble tau is an early pathological event in neurodegeneration, which is also associated with the aging process. The normal aging process can be associated with acetylated tau, for example, primary age-related tauopathy (PART), formerly called tangle predominant senile dementia (TPSD), is a common pathology associated with human aging where aging brains with neurofibrillary tangles in the aging brain are indistinguishable from those of Alzheimer's disease. See, Irwin et al., Am J Pathol. 2013 August: 183 (2): 344-51: doi: 10.1016/j.ajpath.2013.04.025: Crary, et al., Acta Neuropathol. 2014 December: 128 (6): 755-66: doi: 10.1007/s00401-014-1349-0); Sepulcre, et al., J Neurosci. 2016 Jul. 13: 36 (28): 7364-74: doi: 10.1523/JNEUROSCI.0639-16.2016. In an aspect, the normal aging process is of a subject with Down's Syndrome (DS) or amyotrophic lateral sclerosis (ALS), a common pathology in the aging brain of these subjects. See, National Institute on Aging. “Scientists map genome regions that regulate speed of brain aging.” Aug. 25, 2022. In some embodiments, the subject is a research subject, e.g., a laboratory animal. In other embodiments, the subject is one that has been diagnosed with a disorder associated with acetylated tau. In another embodiment, the subject may be one that is at risk of developing a disorder associated with acetylated tau (e.g., predisposed due to hereditary factors, exposure to a pathogen, etc.). Disorders associated with acetylated tau include, without limitation, Alzheimer's disease, Parkinson's disease, Parkinson's disease with dementia, dementia with Lewy bodies, amyotrophic lateral sclerosis, Down's syndrome, corticobasal degeneration, frontal temporal dementia, Pick's disease, multisystem atrophy, progressive supranuclear palsy, inclusion body myositis, prion protein cerebral amyoid angiopathy, argyrophilic grain disease, tangle predominant dementia, chronic traumatic encephalopathy, and traumatic brain injury.

The antibody or antigen binding fragment compositions of the present invention may be administered via any pharmaceutically acceptable routes of administration. Systemic administration, methods of administering to a subject via the bloodstream, is contemplated in accordance with the invention. Localized routes of administration, e.g, administration of the composition at the site where the desired action is required, are also contemplated. Routes of administration include enteral, parenteral and inhalation routes.

Example routes of administration include intranasal, intramuscular, intratracheal, intrathecal, subcutaneous, intradermal, topical, intravenous, intraarterial, rectal, nasal, oral, and other enteral and parenteral routes of administration. The routes of administration can be combined, if desired, or adjusted depending on the desired effect and composition utilized. In an embodiment, the route of administration is parenteral administration, including intravenous, intramuscular, intradermal, intraperitoneal, rectal, intranasal, urogenital, otic, dermal, topical, buccal, vaginal, nasal, sublingual, subcutaneous, spinal cord administration or other parenteral routes of administration, for example by injection or infusion, e.g. intravenously, intramuscularly, intraarterial, subepidural intraperitoneal, intracapsular, intraocular, intracardiac, intradermal, transtracheal, subcutaneous, subepithelial, subcapsular, submucosal, intraspinal, epidural and intrathoracic injections and infusions.

Enteral administration, including oral and rectal administration is contemplated. Administration of the compounds of the present invention may be carried out by any form of oral administration by a tablet, a pill, a capsule, a granule, a powder, a solution, or the like. In some embodiments of the presently-disclosed subject matter, the antibody, or antigen binding fragment thereof, can be provided in an ingestible vehicle, appropriate for administration, particularly oral administration, to the subject. Any ingestible vehicle appropriate for delivering an antibody to a subject as is known to those of ordinary skill in the art can be used. Example delivery of antibodies by oral administration can include ligand-conjugated lipid particles. See, e.g., Shreya et al., AAPS ParmSciTech (2019) 20:15: doi: 10.1208/s12249-018-1262-2. Similarly, compositions comprising the antibody, or antigen binding fragment thereof, can be provided for rectal administration in a suppository.

A further aspect of the invention is a method of treating subjects in vivo, comprising administering to a subject a pharmaceutical composition comprising a monoclonal antibody or an antigen binding fragment thereof. Administration of the monoclonal antibody or an antigen binding fragment thereof of the present invention to a human subject or an animal in need thereof can be by any means known in the art for administering compounds.

The antibodies of the present invention can optionally be delivered in conjunction with other therapeutic agents. The additional therapeutic agents can be delivered concurrently with the antibodies of the invention. As used herein, the word “concurrently.” means sufficiently close in time to produce a combined effect (that is, concurrently can be simultaneously, or it can be two or more events occurring within a short time period before or after each other).

In one embodiment, the antibodies of the invention are administered in conjunction with anti-AD agents such as, for example, phospho-tau or amyloid beta immunotherapy antibodies (e.g. Aducanumab), a cholinesterase inhibitor (e.g. Aricept), or a glutamate regulator (e.g. Namenda).

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa). The therapeutically effective dosage of any specific compound will vary somewhat from compound to compound, and patient to patient, and will depend upon the condition of the patient and the route of delivery. As a general proposition, a dosage from about 0.001 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of a tauopathy such as AD. In certain embodiments, the subject has or is at risk for a tauopathy such as AD.

A monoclonal antibody or an antigen binding fragment thereof as disclosed herein may be used for detection or diagnostic purposes in vivo and/or in vitro. For example, a wide range of immunoassays involving antibodies for detecting the expression in specific cells or tissues are known to the skilled person.

For such applications the monoclonal antibody or an antigen binding fragment thereof, e.g, the antibody disclosed herein, may include a detectable label. In some embodiments the monoclonal antibody or an antigen binding fragment thereof disclosed herein does not include a detectable label. As an illustrative example, an unlabelled antibody may be used and detected by a secondary antibody specifically binding to an epitope on the monoclonal antibody or an antigen binding fragment thereof, e.g, antibody, described herein.

In some embodiments the monoclonal antibody or an antigen binding fragment thereof is coupled to one or more substances that can be recognized by a detector substance. As an example, the monoclonal antibody or an antigen binding fragment thereof may be covalently linked to biotin, which can be detected by means of its capability to bind to streptavidin. Likewise, the nucleic acids and/or vectors disclosed herein can be used for detection or diagnostic purposes, e.g., by using labelled fragments thereof as probes in hybridization assays.

In certain embodiments, any of the molecules provided herein, in particular the antibody, is useful for detecting the presence of acetylated tau in a sample, preferably a sample of biological origin. The term “detecting” encompasses quantitative and/or qualitative detection. In certain embodiments a biological sample includes a cell or tissue from human patients. Non limiting examples of biological samples include blood, urine, cerebrospinal fluid, biopsy, lymph and/or non-blood tissues.

In certain embodiments, the method includes contacting the biological sample with a monoclonal antibody or an antigen binding fragment thereof to acetylated tau (such as an anti-acetylated tau antibody) as described herein under conditions permissive for binding of the inhibitor to its target acetylated tau, if present, and detecting the inhibitor-target complex. Such method may be an in vitro or in vivo method. In one embodiment such monoclonal antibody or an antigen binding fragment thereof is used to select subjects eligible for therapy with the monoclonal antibody or an antigen binding fragment thereof described herein, e.g., where acetylated tau is a biomarker for selection of patients.

As one aspect, the invention provides methods of diagnosing a disease associated with a tau protein acetylated at lysine 280 and/or lysine 311 in a subject, the method comprising contacting a biological sample from the subject with the monoclonal antibody or an antigen binding fragment thereof under conditions permissive for specific binding of the monoclonal antibody or an antigen binding fragment thereof to tau protein acetylated at lysine 280 and/or lysine 311 and detecting whether a complex between the monoclonal antibody or an antigen binding fragment thereof and tau protein acetylated at lysine 280 and/or lysine 311 is formed, wherein detection of the complex indicates the onset or presence of a condition or age-related disease associated with a tau protein acetylated at lysine 280 and/or lysine 311 in the subject. Furthermore, detection of a complex at lysine 280 and not at lysine 311 or at lysine 311 and not at lysine 280 or detection of a complex at both lysine 280 and lysine 311 may further indicate the onset or presence of a subset of conditions or age-related diseases associated with an acetylated tau protein. For example, acetylation at lysine 280 and not at lysine 311 may be indicative of corticobasal degeneration, whereas acetylation at lysine 311 and not at lysine 280 may be indicative of Pick's disease. Acetylation at both lysine 280 and lysine 311 may be indicative of Alzheimer's disease. Further diagnostic testing may be desirable and/or required to further differentiate the subsets of tauopathies and/or confirm the presence of a tauopathy. In an embodiment, detection of any complex between the monoclonal antibody or an antigen binding fragment thereof, indicates tauopathy, as the normal brain lacks tauopathy and would have no detectable pathology or acetylated tau. In an embodiment, a 1%, 2%, 3%, 4%, 5%, 6%, 7%, 8%, 9%, 10% or more increase over baseline in detectable complex between the monoclonal antibody, or an antigen binding fragment thereof, is present.

As one aspect, the invention provides a monoclonal antibody targeting acetylated tau or an antigen binding fragment thereof, the nucleic acid molecule encoding the monoclonal antibody, or an antigen binding fragment thereof, the vector containing the nucleic acid sequence, or the host cell containing the monoclonal antibody, nucleic acid sequence or vector for use as a medicament, in particular in the treatment of a disease or condition associated with a tau protein acetylated at lysine 280 and/or lysine 311, for use as a medicament, in particular in the prevention or delay in the progression of the normal aging process associated with a tau protein acetylated at lysine 280 and/or lysine 311, for use in diagnostics, and/or for detection purposes.

A further aspect of the invention relates to kits for use in the methods of the invention. The kit can comprise the antibody of the invention in a form suitable for administration to a subject or in a form suitable for compounding into a formulation. The kit can further comprise other therapeutic agents, carriers, buffers, containers, devices for administration, and the like. The kit can further comprise labels and/or instructions, for treatment of a disorder. Such labeling and/or instructions can include, for example, information concerning the amount, frequency and method of administration of the antibody.

The kit can comprise the antibody, or antigen binding fragment thereof, of the invention in a form suitable for diagnostic use, or suitable for compounding into a diagnostic or detection formulation. The detection compositions can be formulated for administration to a subject or in vitro use with a biological sample. The kit can further comprise other labeling agents, solid supports, carriers, buffers, containers, devices for administration, and the like. The kit can further comprise labels and/or instructions, for detection of a disorder. Such labeling and/or instructions can include, for example, information concerning measurement amount, background corrections, and method of administration of the antibody.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### Example 1

### Generation, Purification, and Validation of Monoclonal Antibodies

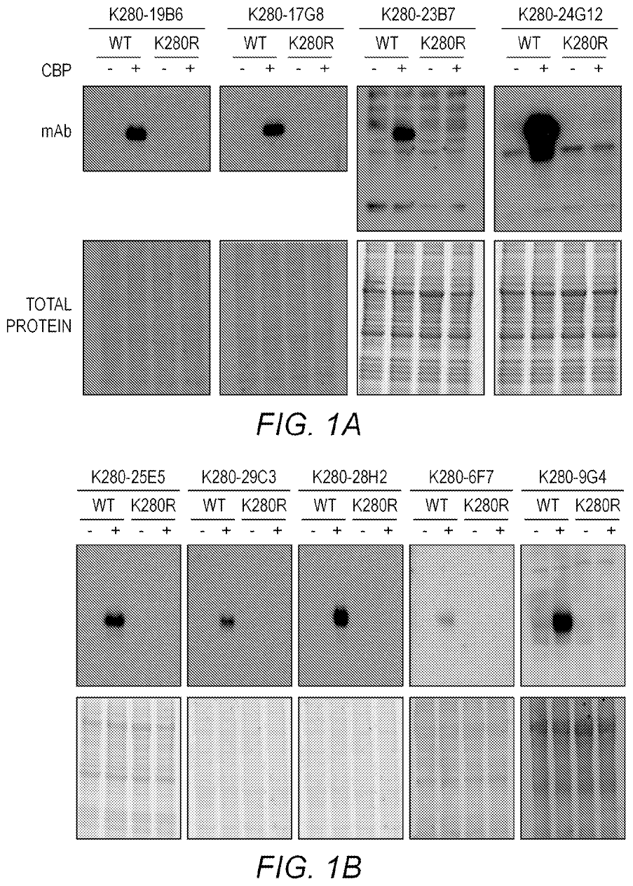

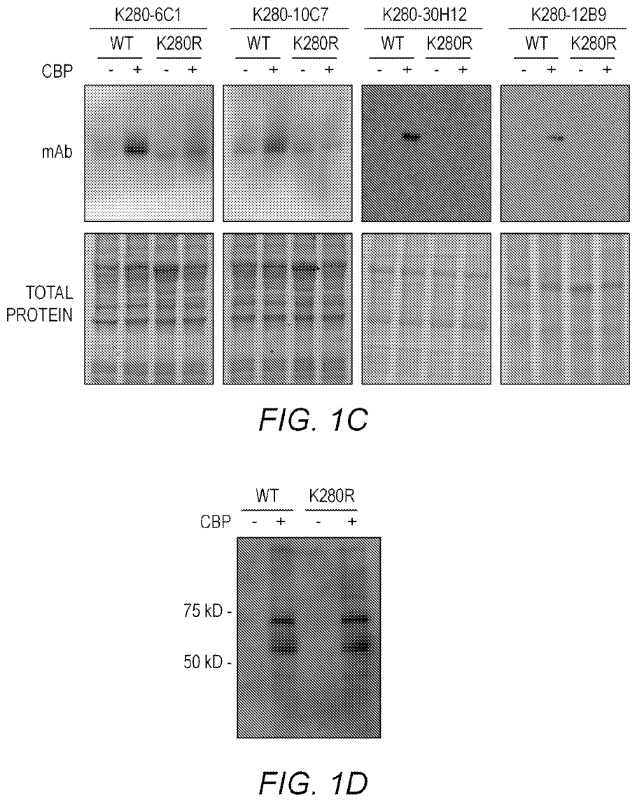

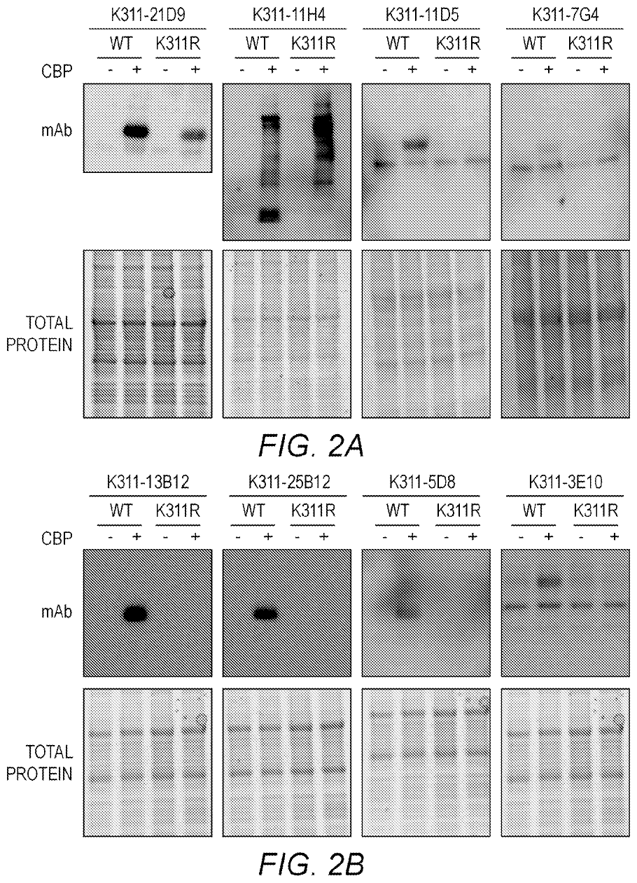

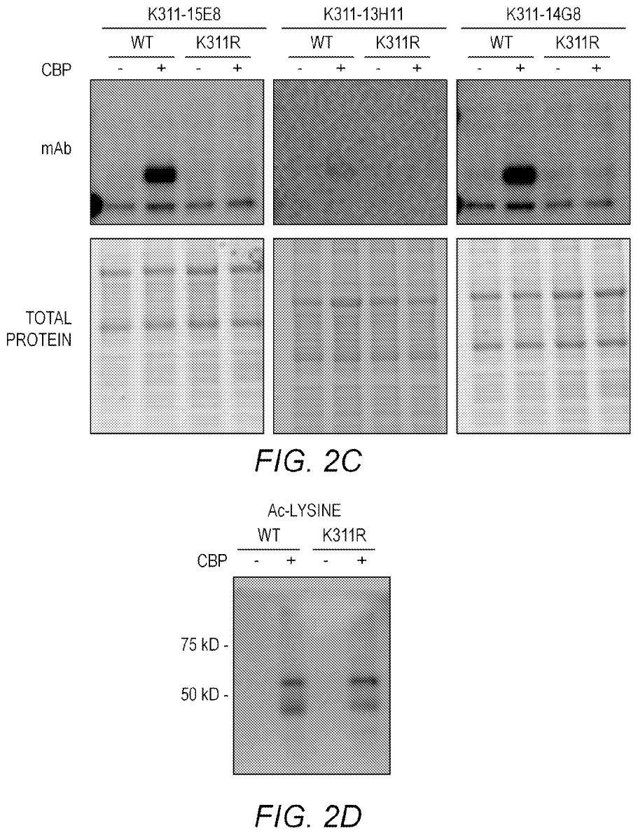

Thousands of mouse monoclonal antibodies were generated against either one of the following acetylated peptides: QIVYKPVDLSKVTSC (SEQ ID NO: 15) (K311) or QIINKKLDLSNVQSC (SEQ ID NO: 16) (K280). The antibodies were purified for downstream applications. 50 μl of protein A/G or G magnetic IgG beads (Pierce) were washed once with 150 μl of NETN buffer, then again with 500 μl of NETN buffer. Then, 500 μl of hybridoma supernatant was thawed and added to the beads overnight at 4° C. The following day, the supernatant was discarded and the beads were washed 3× with 500 μl of NETN buffer. The antibodies were eluted off the beads using low pH or gentle elution buffer (Pierce). If low pH buffer was used, this was neutralized with Tris pH10.4 buffer (1:10). Antibody aliquots were stored at −20° C. Selected antibodies are listed in Table 1 and shown in FIGS. 1A-1D and FIGS. 2A-2D.

HEK293 cells were transfected with full length, wild-type tau or full length. K280R/K311R (acetylation-null) mutant tau with and without the acetyl transferase CREB binding protein (CBP/p300) for 24 hours. Acetylation-null mutants cannot undergo acetylation at their respective residues. Cell lysates were processed for western blot and probed with Ac-tau monoclonal antibodies at a ratio of 1:100 (˜5-10 μg/mL) overnight. Blots were incubated with HRP-linked anti-mouse secondary antibodies and imaged by chemiluminescence.

FIGS. 1A-1D are western blots of cell extracts probed with purified Ac-K280 antibodies in HEK293 cells with and without the acetyl transferase CREB binding protein (CBP/p300). FIGS. 1A-1D show validation that Ac-K280) monoclonal antibodies bind preferentially to acetylated wild-type (WT) K280 compared to non-acetylated K280R mutants in HEK293 cells using western blots. FIGS. 2A-2D are western blots of cell extracts probed with purified Ac-K311 antibodies in HEK293 cells with and without the acetyl transferase CREB binding protein (CBP/p300). FIGS. 2A-2D show validation that Ac-K311 monoclonal antibodies bind preferentially to acetylated wild-type (WT) K311 compared to non-acetylated K31IR mutants in HEK293 cells using western blots. Table 1 is a compiled list of Ac-K311 and Ac-K280 clones validated by western blot (WB) and immunocytochemistry (ICC) after IgG purification from hybridoma supernatant. Target and antibody isotype are listed for each clone. Clones that were completely non-specific or undetectable were not included.

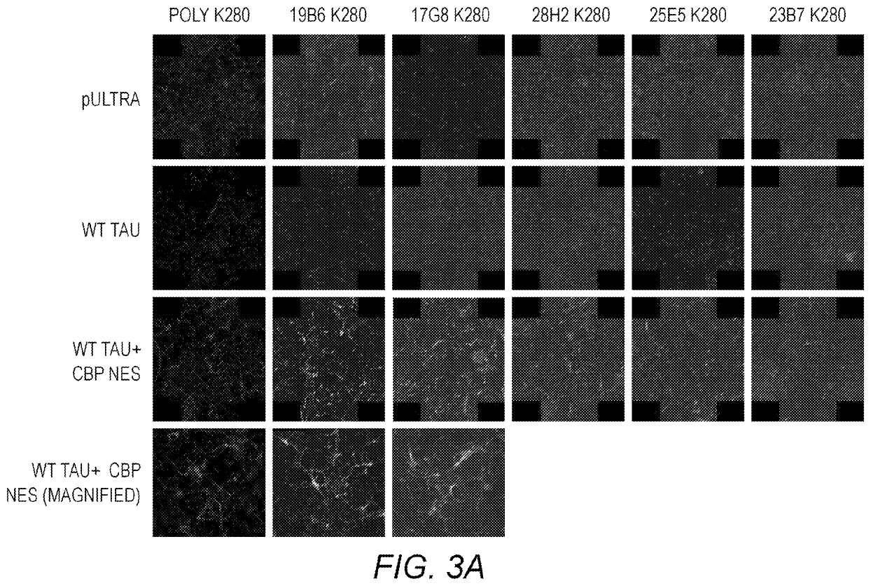

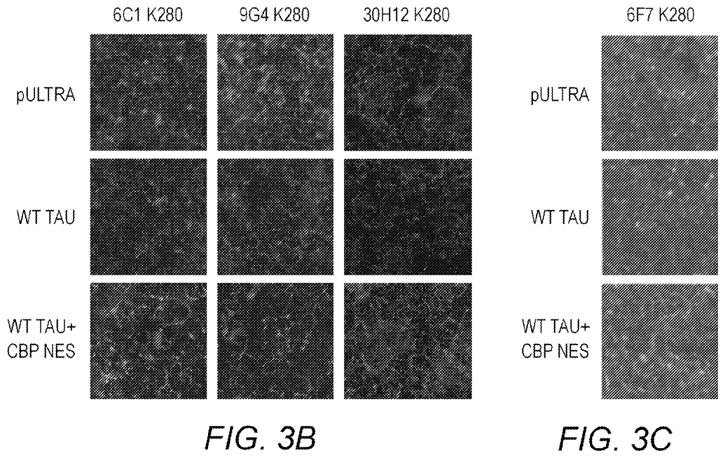

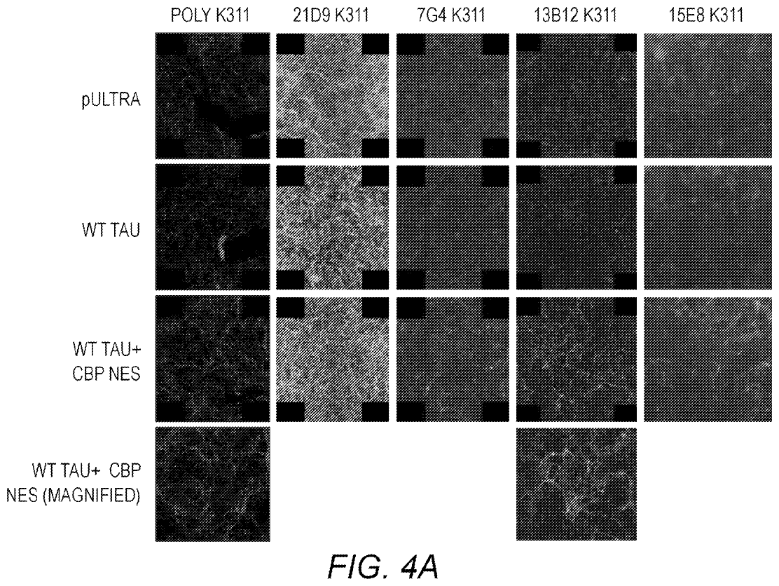

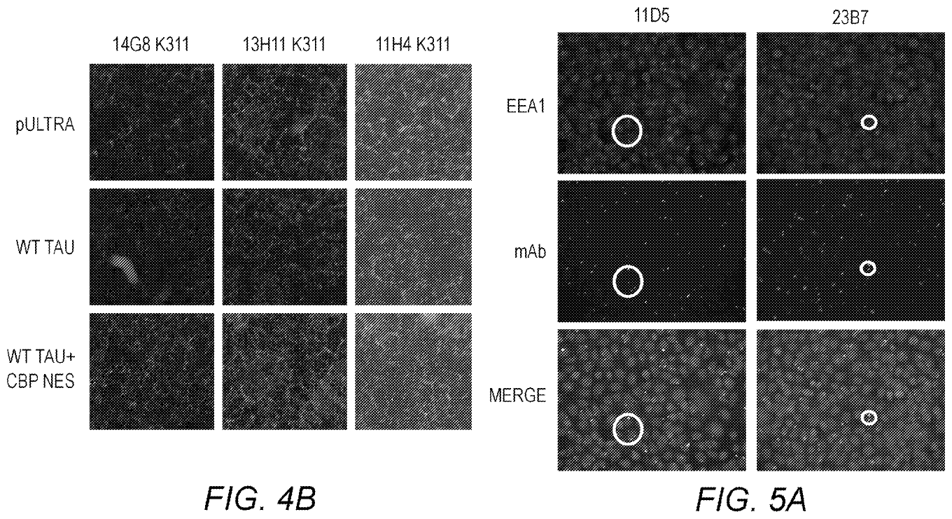

FIGS. 3A-3C are a series of immunocytochemistry images of Ac-K280 antibodies in primary cortical neurons. FIGS. 3A-3C show images with pUltra empty vector as a control, with WT tau, and with WT tau and the catalytic domain of CREB binding protein with a nuclear exclusion sequence. FIGS. 4A-4B are a series of immunocytochemistry images of Ac-K311 antibodies in primary cortical neurons. FIGS. 4A-4B show images with pUltra empty vector as a control, with WT tau, and with WT tau and the catalytic domain of CREB binding protein with a nuclear exclusion sequence. FIGS. 3A-3C and FIGS. 4A-4B were generated by culturing primary cortical neurons from WT C57BL/6 E16 embryos. Neurons were plated onto poly-D-lysine coated 96-well plates at 30.000 neurons/well. At DIV3, neurons were transduced with lentiviral expression vectors 1) pUltra empty vector 2) full length, wild-type tau or 3) full length, wild-type tau with the catalytic domain of CREB binding protein with a nuclear exclusion sequence (this enhances cytoplasmic acetylation). Neurons were cultured until DIV10, fixed with 4% paraformaldehyde, blocked with 4% normal goat serum, and incubated with primary antibodies overnight (1:25 or ˜20-40 μg/mL for monoclonal antibodies, 1:250 for polyclonal antibodies). The following day, neurons were incubated with highly cross-absorbed ALEXA FLUOR® secondary antibodies for two hours. Neurons were then DAPI stained and 5-10% of each well was imaged at 20× and stitched using an automated EVOS m7000 microscope. Previously generated polyclonal (Poly) antibodies (K280 and K311) were included for comparison in AF594 (RFP). Monoclonal antibodies were labeled with AF488 (GFP).

TABLE 1
Purified Monoclonal Antibodies against
acetylated tau at either K311 or K280

| Clone ID | target | Isotype | WB validated | ICC validated |
|---|---|---|---|---|
| 21D9 | K311 | IgG2a, K | ++ | n.s. |
| 11H4 | K311 | IgG1, K | + | + |
| 11D5 | K311 | IgG1, K | ++ | n.s |
| 7G4 | K311 | IgG1, K | + | ++ |
| 13B12 | K311 | IgG2b, K | +++ | +++ |
| 25B12 | K311 | IgG2b, K | +++ | n.s |
| 5D8 | K311 | IgG2b, K | ++ | n.s |
| 3E10 | K311 | IgG1, K | + | n.s |
| 15E8 | K311 | IgG1, K | ++ | ++ |
| 13H11 | K311 | IgG1, K | + | + |
| 14G8 | K311 | IgG1, K | ++ | ++ |
| 12B9 | K280 | IgG2b, K | + | − |
| 19B6 | K280 | IgG1, K | +++ | +++ |
| 17G8 | K280 | IgG1, K | +++ | +++ |
| 23B7 | K280 | IgG2b, K | ++ | + |
| 24G12 | K280 | IgG1, K | ++ | −− |
| 25E5 | K280 | IgG1, K | +++ | +++ |
| 29C3 | K280 | IgG1, K | ++ | − |
| 28H2 | K280 | IgG1, K | +++ | +++ |
| 6F7 | K280 | IgG2b, K | ++ | + |
| 9G4 | K280 | IgG2b, K | ++ | ++ |
| 6C1 | K280 | IgG2b, K | + | +++ |
| 10C7 | K280 | IgG2b, K | + | − |
| 30H12 | K280 | IgG1, K | + | + |

+ = low signal and/or specificity; +++ = high signal and/or specificity; n.s. = non-specific signal; no signal = undetectable, − = not tested.

### Example 2

### Monoclonal Antibodies Delivered with Carrier Molecule Colocalize with EEA1 and Rab5 (Endosomal), and LAMP1 (Lysosomal) in HEK293 Cells

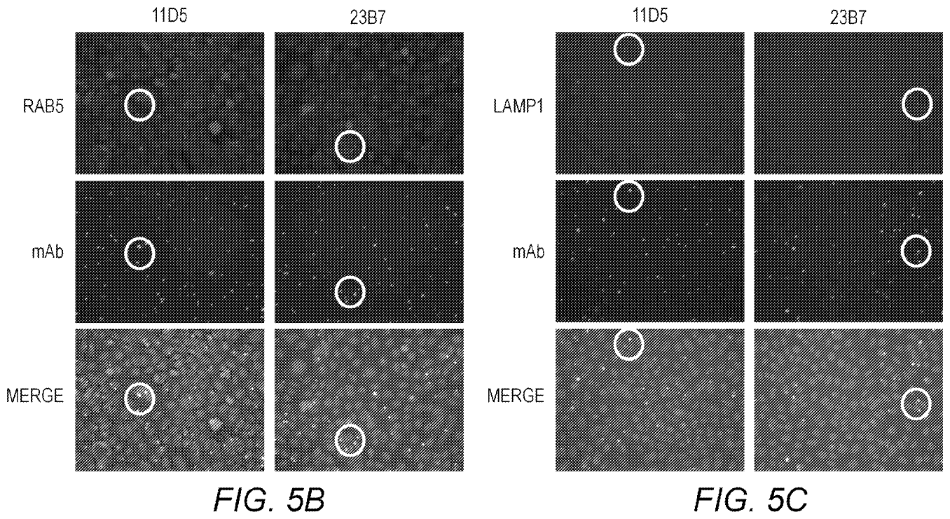

FIGS. 5A-5C are images of monoclonal antibodies delivered with carrier molecule in HEK293 cells. FIGS. 5A-5C show that the monoclonal antibodies 11D5 (Ac-K311) and 23B7 (Ac-K280) colocalize with EEA1 and Rab5 (endosomal), and LAMP1 (lysosomal) indicating the internalization of the antibodies. HEK293 cells were incubated for 24 hrs with a lipophilic carrier molecule (Ab-DeliverIN™ Transfection Reagent, OzBiosciences, 1 μL per reaction and per well) and purified monoclonal antibodies (0.5 μg per reaction and per well). Cells were fixed with 4% paraformaldehyde, blocked with 4% normal donkey serum, and incubated with primary antibodies overnight (1:250). The following day, the cells were washed and incubated with secondary antibodies for 2 hrs (AF549 (RFP)-EEA1, Rab5, LAMP1) and anti-mouse AF488 (GFP) which detects the internalized mouse Ac-tau monoclonal antibodies. Select images for monoclonal antibody clones 11D5 (Ac-K311) and 23B7 (Ac-K280) are shown. White circles denote areas of colocalization.

### Example 3

### Monoclonal Antibodies are Taken Up by Neurons and Colocalize with Ac-Tau Varicosities

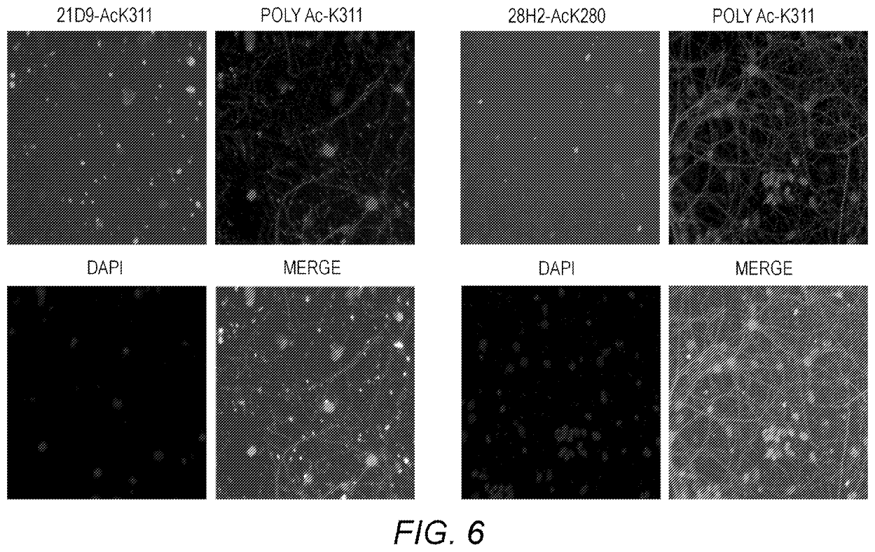

FIG. 6 is a series of images of monoclonal antibodies in primary cortical neurons. FIG. 6 shows that the monoclonal antibodies are taken up by the primary cortical neurons and colocalize with Ac-tau varicosities. Primary cortical neurons were cultured as in FIGS. 3A-3C and FIGS. 4A-4B. At DIV7, neurons were incubated with lug/mL of monoclonal antibody (no carrier molecule was used) for 72 hours. Neurons were prepared for ICC-monoclonal antibodies labeled with AF488 (GFP): polyclonal Ac-K311 labeled with AF594 (RFP); DAPI (BFP). Neurons were imaged at 20× with an EVOS m700 microscope. Select images for monoclonal antibody clones 21D9 (Ac-K311) and 28H2 (Ac-K280) are shown.

### Example 4

### Monoclonal Ac-K280 Mabs Detect Amyloid Plaques and Neurofibrillary Tangles in AD Post-Mortem Brain

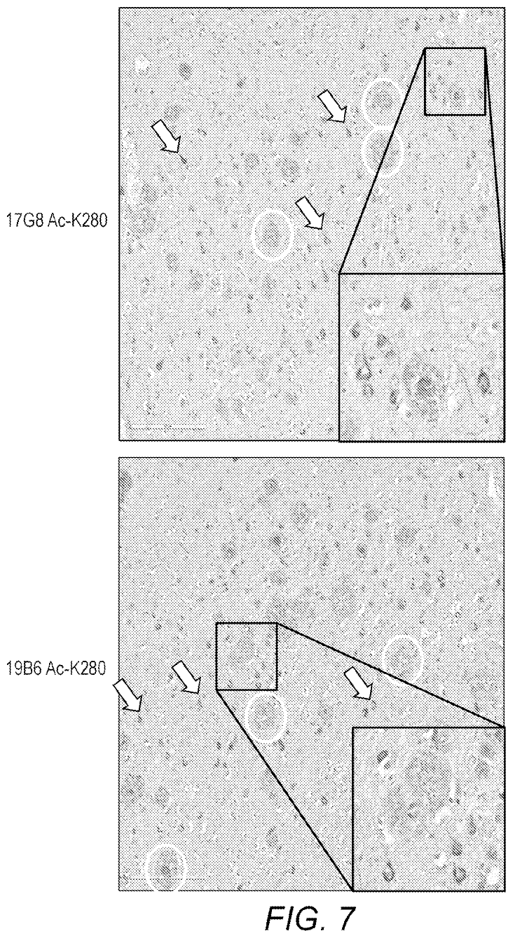

FIG. 7 is a series of monoclonal antibody images in Alzheimer disease (AD) post-mortem brain. FIG. 7 shows that the Ac-K280 monoclonal antibodies are able to detect amyloid plaques and neurofibrillary tangles in Alzheimer disease post-mortem brains. Human, Braak VI AD post-mortem brain was provided by the Center for Neurodegenerative Disease Research (CNDR) at the University of Pennsylvania. Sections were stained with monoclonal Ac-tau K280 antibodies (19B6 and 17G8) at 1:25 and visualized using an avidin-biotin complex detection system. Sections were lightly counterstained with hematoxylin and then imaged. White circles are representative amyloid plaques and arrows are representative neurofibrillary tangles.

Together, these data support that several of these clones (either K280 or K311) exhibit various biochemical (sensitivity and specificity) and biological (proper antibody trafficking, binding to Ac-tau in living cells) properties which make them excellent candidates for: 1) Ac-tau-directed immunotherapy and 2) diagnostic tools to detect Ac-tau in AD models and patients via ELISA.

The refined candidates include several different isotypes (IgG1, K: IgG2a,K: IgG2b,K). Several antibodies, 11 Ac-K280 and 8 Ac-K311, were validated and showed that they were highly specific for their specific acetylated residues by western blot (FIGS. 1A-1D and FIGS. 2A-2D, Table 1). Many of these antibodies, 5 Ac-K280 and 4 Ac-K311, were validated to detect acetylated tau induced in cortical primary neuron cultures (FIGS. 3A-3C and FIGS. 4A-4B). When these antibodies were delivered with a carrier molecule (Ab-DeliverIN™) in cell culture (HEK293), it was observed that they colocalize with early endosomal compartments (EEA1), suggesting the appropriate trafficking necessary for tau clearance and neuronal immunotherapeutic potential (FIG. 5A). Applicant has found that several of these antibodies are internalized (without the need of exogenous carrier) in primary cortical neurons, in particular clone 21D9 (Ac-K311), and internalized antibodies co-localized with CBP-induced acetylated tau (FIG. 6). Antibody candidates that are not internalized into neurons may still exhibit exquisite immunotherapeutic potential by binding extracellular tau, inhibiting its ability to be transmitted to nearby neurons, and promoting its degradation by microglia.

### Example 5

### Monoclonal Ac-K280 and Ac-K311 Mabs Detect Tau Pathology

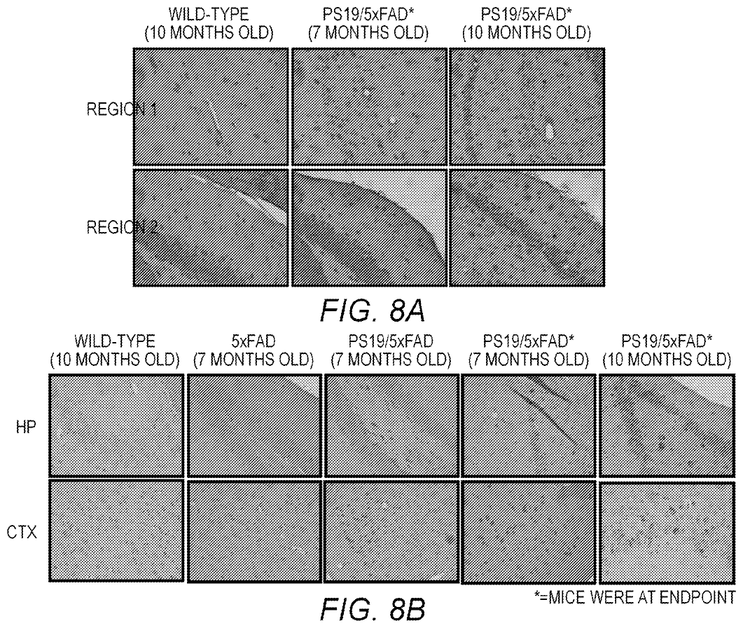

FIGS. 8A-8B are a series of immmunohistochemistry images of mouse brain regions. FIG. 8A shows Ac-K280) clone 19B6 detects tau pathology in PS19/5×FAD mice. Immunohistochemistry was performed on aged PS19×5×FAD mice 3,3-diaminobenzidine (DAB) immunostained with monoclonal Ac-K280 clone 19B6 at a 1:250 dilution. Images were taken from three mice, one wild-type and two PS19/5×FAD mice. Two hippocampal regions were included per mouse. *=mice were sacrificed because they had reached end-point pathology. FIG. 8B shows that Ac-K280 tau is present in aged PS19/5×FAD mice. Immunohistochemistry was carried out on aged PS19×5×FAD mice using Ac-K280 (polyclonal). Images are taken from 5 mice of varying age (7-10 months) and pathology (wild-type, 5×FAD, PS19/5×FAD). Hippocampal and cortical images are both shown showing Ac-K280 inclusions in PS19/5×FAD mice only.

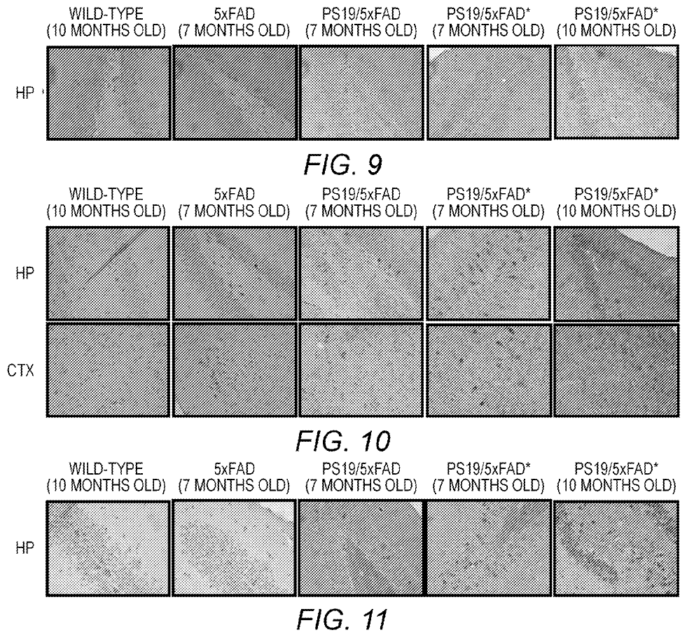

Immunohistochemistry on aged PS19×5×FAD mice for Ac-K311 shows Ac-K311 tau is present (polyclonal) (FIG. 9). Images were taken from 5 mice of varying age (7-10 months) and pathology (wild-type, 5×FAD, PS19/5×FAD). Hippocampal images shown with Ac-K311 positive neuritic threads are present in PS19/5×FAD mice only. FIG. 10 images show phosphorylated tau is present in aged PS19/5×FAD mice. Immunohistochemistry was conducted on aged PS19×5×FAD mice for AT8, with images taken from 5 mice of varying age (7-10) months) and pathology (wild-type, 5×FAD, PS19/5×FAD). Hippocampal (Hp) and cortical (Ctx) images are shown.

Immunochemistry was conducted on aged PS19×5×FAD mice for MCI showing conformational tau is present in aged PS19/5×FAD mice. Images in FIG. 11 were taken from 5 mice of varying age (7-10 months) and pathology (wild-type, 5×FAD, PS19/5×FAD). Both hippocampal (Hp) and cortical (Ctx) images are shown.

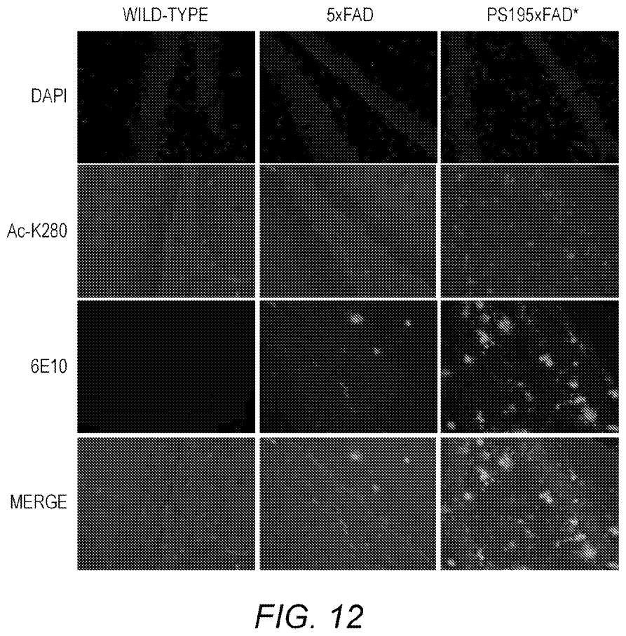

Ac-K280 and amyloid beta plaques accumulates in aged PS19/5×FAD mice (FIG. 12). Fluorescent immunohistochemistry on the hippocampus of aged mice for Ac-K280 (polyclonal) and 6E10 (amyloid beta). *=mice were sacrificed because they had reach end-point pathology. PS19/5×FAD (and to a lesser extent, 5×FAD) mice exhibit intracellular Ac-K280 and extracellular amyloid beta plaques which are not present in wild-type mice.

### Example 6

### Monoclonal K280 Mabs Preferentially Detect Acetylated Tau Peptides

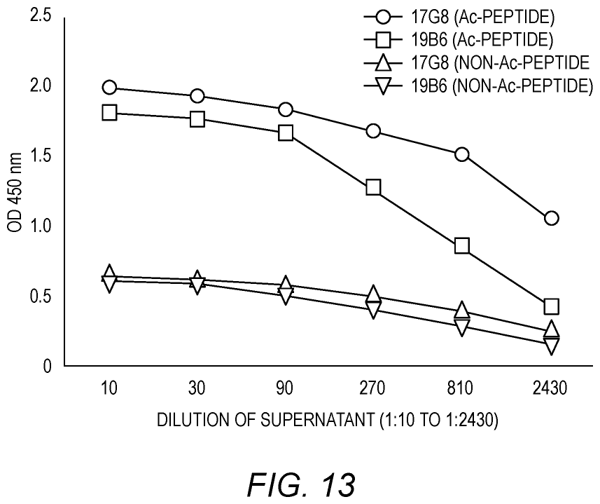

FIG. 13 includes an ELISA assay showing two K280 clones, 19B6 and 17G8, preferentially detect acetylated vs, non-acetylated tau peptides. Standard peptide ELISAs were performed with 1 μg/ml acetylated peptides at residue K280 (276QIIN-K-Ac-KLDLSNVQSC (SEQ ID NO:17)) vs, non-acetylated peptides (276QIINKKLDLSNVQSC (SEQ ID NO: 16)) and used to coat 96-well plates followed by washing with coating buffer (Phosphate Buffered Saline, pH 7.4) and incubated with serial dilutions of hybridoma supernatants (diluted from 1:10 to 1:2,500). Monoclonal antibodies were conjugated to Horseradish peroxidase (HRP)-labeled secondary antibodies and colorimetric ELISAs were performed using a BMG/BioTek® multiplate reader at a standard absorbance setting of 450 nm with auto-mixing and blanking.

### Example 7

### Monoclonal Ac-K280 and Ac-K311 Mabs Detect Amyloid Plaques and Neurofibrillary Tangles in AD Post-Mortem Brain

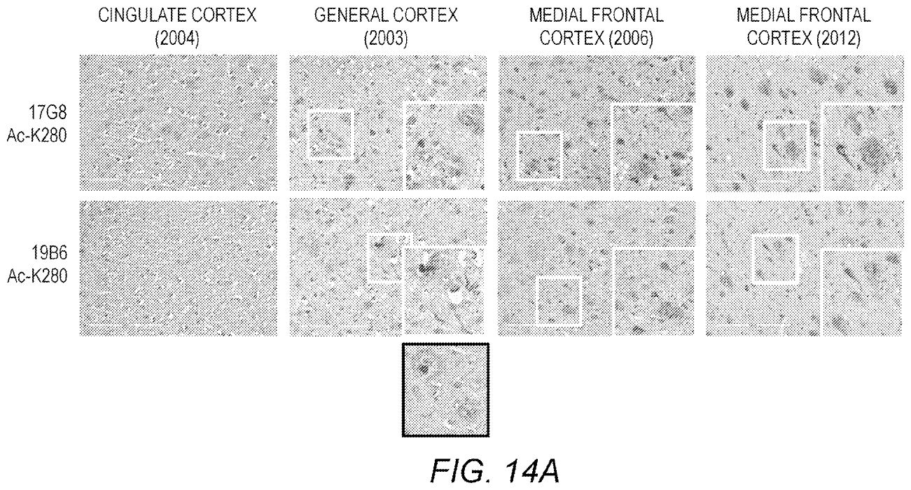

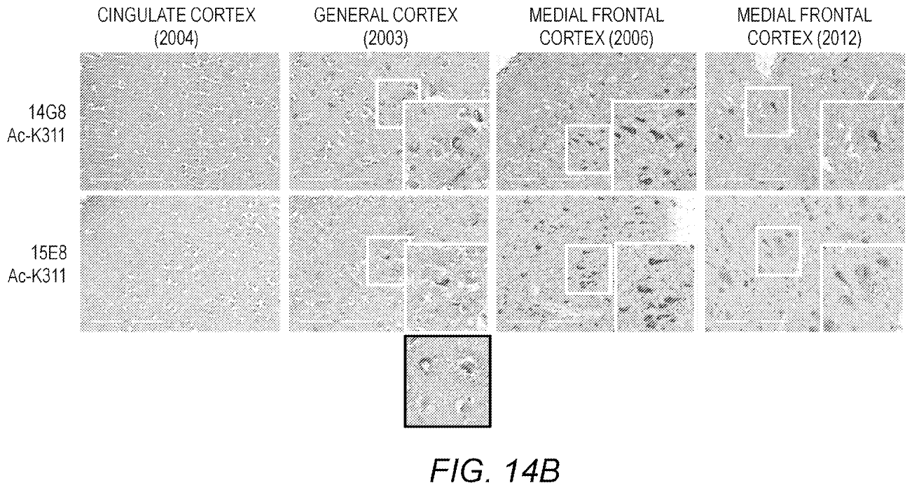

FIGS. 14A and 14B are a series of monoclonal antibody images in Alzheimer disease (AD) post-mortem brain. FIG. 14A shows that the Ac-K280 monoclonal antibodies are able to detect amyloid plaques and neurofibrillary tangles in Alzheimer disease post-mortem brains. FIG. 14B shows that the Ac-K311 monoclonal antibodies are able to detect amyloid plaques and neurofibrillary tangles in Alzheimer disease post-mortem brains. Human, Braak VI AD post-mortem brain was provided by the Center for Neurodegenerative Disease Research (CNDR) at the University of Pennsylvania. Sections were stained with monoclonal Ac-tau K280 antibodies (Ac-K280 clones 19B6 and 17G8 (FIG. 14A); Ac-K311 14G8 and 15E8 (FIG. 14B)) at 1:25 and visualized using an avidin-biotin complex detection system. Sections were lightly counterstained with hematoxylin and then imaged. Cortical brain sections (across three different cortical regions) from four separate AD patients were stained with both sets of Ac-K280 and Ac-K311 clones. White square represents magnified inset. Additional image of neurofibrillary tangle closely associated with amyloid beta plaques are included at the bottom of figure (black border inset).

### Example 8

### Monoclonal Antibody Sequences for K280 and K311 Clones

Monoclonal antibody sequences for K280 clones and K311 clones are provided below. Each antibody clone heavy and light chain (kappa) were sequenced following the methods of L. Meyer, R. DuBois et al, 2019 using the SMARTer RACE 5′/3′ kit (Takara). Notably, many clones share highly similar heavy chain sequences, but variable kappa light chain sequences which dictate efficacy in detecting Ac-tau by western blot, Immunocytochemistry (ICC), and Immunohistochemistry (IHC).

### Sequences

### K280 Monoclonal Antibody Sequences

19B6 Heavy
(SEQ ID NO: 18)
EVQLQQSGAEFVRPGASVKLSCTVSGLNIEDDYIQWLKQRPEQGLEWIG
WIDPENGDPKYASKFQGKATLTADTSSKIAYLHLSSLTSEDTAVYYCAT
RGLWGQGTTLTVSS
19B6 Kappa
(SEQ ID NO: 19)
QIVLTQSPAIMSASLGERVTMTCTASSSVSSSYFHWYQQKPGSSPKLWI
YSTSNLASGVPVRFSGSGSGTSYSLTISSMEAEDAATYYCHQYHRSPLT
FGAGTKLELK
17G8 Heavy
(SEQ ID NO: 20)
EVQLQQSGAEFVRPGASVKLSCTVSGLNIEDDYIQWLKQRPEQGLEWIG
WIDPENGDPKYASKFQGKATLTADTSSKIAYLHLSSLTSEDTAVYYCAT
RGLWGQGTTLTVSS
17G8 Kappa
(SEQ ID NO: 21)
QIVLTQSPAIMSASLGERVTMTCTASSSVSSSYFHWYQQKPGSSPKLWI
YSTSNLASGVPVRFSGSGSGTSYSLTISSMEAEDAATYYCHQYHRSPLT
FGAGTKLELK
25E5 Heavy
(SEQ ID NO: 22)
EVQLQQSGAEFVRPGASVKLSCTVSGLNIEDDYIQWLKQRPEQGLEWIG
WIDPENGDPKYASKFQGKATLTADTSSKIAYLHLSSLTSEDTAVYYCAT
RGLWGQGTTLTVSS
25E5 Kappa
(SEQ ID NO: 23)
QIVLTQSPAIMSASLGERVTMTCTASSSVSSSYFHWYQQKPGSSPKLWI
YSTSNLASGVPVRFSGSGSGTSYSLTISSMEAEDAATYYCHQYHRSPLT
FGAGTKLELK
28H2 Heavy
(SEQ ID NO: 24)
EVQLQQSGAEFVRPGASVKLSCTVSGLNIEDDYIQWLKQRPEQGLEWIG
WIDPENGDPKYASKFQGKATLTADTSSKIAYLHLSSLTSEDTAVYYCAT
RGLWGQGTTLTVSS
28H2 Kappa
(SEQ ID NO: 25)
QIVLTQSPPIMSASLGERVTMTCTASSSVSSSYFHWYQQKPGSSPKLWI
YSTSNLASGVPVRFSGSGSGTSYSLTISTMEAEDAATYYCHQYHRSPLT
FGAGTKLELK
9G4 Heavy
(SEQ ID NO: 26)
EVRLQQSGAEFVKPGASVKLSCTSSGFNIKDYYMHWVKQRTEQGLEWIG
MIDPENGETKYAPKFQGKATITADTSSNTAYLHLSSLTSEDTAVYYFIR
DFEVWGTGTTVTVSS
9G4 Kappa
(SEQ ID NO: 27)
DVLMTQTPLSLPVSLGDQASISCRSSQNIVHGSGHTYLEWYLQMPGQSP
KLLIHKVSNRLSGVPDRFSGSGSGTDFTLKISRVEAEDLGVYYCFQGSH
VPLTFGAGTKLELR
23B7 Heavy
(SEQ ID NO: 28)
EVQLQQSGAELVRPGASVKLSCTASGLNIKDDYMQWIKQRPEQGLEWIG
WIDPENGDTKYASKFQGKATITADTSSNTAYLQLSSLTSEDTAVYFCAT
RGLWGQGTTLTVSS
23B7 Kappa
(SEQ ID NO: 29)
QIVLTQSPAIMSASLGERVTMTCTASSSVSSSYFHWYQQKPGSSPKLWI
YSTSNLASGVPARFSGSGSGTSYSLTISSMEAEDAATYYCHQYHRSPLT
FGAGTKLELK
6C1 Heavy
(SEQ ID NO: 30)
EVQLQQSGAELVRPGASVKLSCTTSGLNIKDDYVNWVKQRPEQGLEWIG
WIDPENDDTKYASKFQGKATITAATSSSTAYLHLNSLTSEDSAVYYCAT
RGVWGQGTTLTVSS
6C1 Kappa
(SEQ ID NO: 31)
QIVLTQSPAIMSASLGERVTMTCTASSSISSTFFHWYQQKTGSSPKLWI
YRTSNLAPGVPARFSGSGSGTSYSLTISNMEAEDTATYYCHQYHRSPLT
FGAGTKLELK
6F7 Heavy
(SEQ ID NO: 32)
EVQLQQSGAELVKPGASVKLSCTTSGFNIKDYFMHWVKQRTEQGLEWIG
MIDPEDGKTKYAPKFQGKATITADPSSNTAYLHLSSLTSEDTAVYYIIR
DFEVWGTGTTVTVSS
6F7 Kappa
(SEQ ID NO: 33)
DVLMTQTPLSLPVSLGDQASISCRSSQSIVHDNGHTYLEWYLQMPGQSP
KLLIHKVSNRLSGVPDRFSGSGSGTDFTLKINRVEAEDLGVYYCFQGSH
IPLTFGAGTKLELK
12B9 Heavy
(SEQ ID NO: 34)
EVRLQQSGAEFVKPGASVKLSCTSSGFNIKDYYMHWVKQRTEQGLEWIG
MIDPENGETKYAPKFQGKATITADTSSNTAYLHLSSLTSEDTAVYYFIR
DFEVWGTGTTVTVSS
12B9 Kappa
(SEQ ID NO: 35)
DVLMTQTPLSLPVSLGDQASISCRSSQNIVHGSGHTYLEWYLQMPGQSP
KLLIHKVSNRLSGVPDRFSGSGSGTDFTLKISRVEAEDLGVYYCFQGSH
VPLTFGAGTKLELR
24G12 Heavy
(SEQ ID NO: 36)
EVQLQQSGAEFVRPGASVKLSCTVSGLNIEDDYIQWLKQRPEQGLEWIG
WIDPENGDPNYASKFQGKATLTADTSSKIAYLHLSSLTSEDTAVYYCAT
RGLWGQGTTLTVSS
24G12 Kappa
(SEQ ID NO: 37)
QIVLTQSPAIMSASLGERVTMTCTASSSVSSSYFHWYQQKPGSSPKLWI
YSTSNLASGVPVRFSGSGSGTSYSLTISSMEAEDAATYYCHQYHRSPLT
FGAGTKLELK
29C3 Heavy
(SEQ ID NO: 38)
EVQLQQSGAEFVRPGASVKLSCTVSGLNIEDDYIQWLKQRPEQGLEWIG
WIDPENGDPKYASKFQGKATLTADTSSKIAYLHLSSLTSEDTAVYYCAT
RGLWGQGTTLTVSS
29C3 Kappa
(SEQ ID NO: 39)
QIVLTQSPAIMSASLGERVTMTCTASSSVSSSYFHWYQQKPGSSPKLWI
YSTSNLASGVPVRFSGSGSGTSYSLTISSMEAEDAATYYCHQYHRSPLT
FGAGTKLELK
30H12 Heavy
(SEQ ID NO: 40)
EVQLQQSGAELVRPGASVKLSCTVSGLNIKDDYIHWVKQRPEQGLEWIG
WIDPENGDNEYASKFQGKATITADTSSKTAYLQFSSLTSEDTAVYYCTT
RGLWGQGTTLTVSS
30H12 Kappa
(SEQ ID NO: 41)
QIVLTQSPAIMSASLGERVTMTCTASSSVSSSYLLWFQQRPGSSPKLWI
YSTSNLASGVPARFSGSGSGTSYSLTISSMEAEDAATYYCHQYRRSPLT
FGAGTKLELK
10C7 Heavy
(SEQ ID NO: 42)
EVRLQQSGAEFVKPGASVKLSCTSSGFNIKDYYMHWVKQRTEQGLEWIG
MIDPENGETKYAPKFQGKATITADTSSNTAYLHLSSLTSEDTAVYYFIR
DFEVWGTGTTVTVSS
10C7 Kappa
(SEQ ID NO: 43)
DVLMTQTPLSLPVSLGDQASISCRSSQNIVHGSGHTYLEWYLQMPGQSP
KLLIHKVSNRLSGVPDRFSGSGSGTDFTLKISRVEAEDLGVYYCFQGSH
VPLTFGAGTKLELR
K311 monoclonal antibody sequences
14G8 Heavy
(SEQ ID NO: 44)
EVQLQQSGAELVRPGSSVKMSCKTSGYTFTNYGLNWVKQRPGQGLEWIG
YIYIGNGYTDYNENFKGKATLTSDTSSSTAYMQLSSLTSEDSAIYFCAR
WRPGYFFDYWGQGTTLTVSS
14G8 Kappa
(SEQ ID NO: 45)
DVLMTQTPLSLPVGLGDQASISCRSSQNIVHSNGDTYLEWYLLKPGQSP
QLLIYKVSNRFSGVPDRFSGGGSGTDFILKISRVEAEDLGVYYCFQSSL
VPLTFGAGTKLELK
15E8 Heavy
(SEQ ID NO: 46)
EVQLQQSGAELVRPGSSVKMSCKTSGYTFTNYGLNWVKQRPGQGLEWIG
YIYIGNGYTDYNENFKGKATLTSDTSSSTAYMQLSSLTSEDSAIYFCAR
WRPGYFFDYWGQGTTLTVS
15E8 Kappa
(SEQ ID NO: 47)
DVLMTQTPLSLPVSLGDQASISCRSSQNIVHTNGDTYLEWYLLKPGQSP
QLLIYKVSNRFSGVPDRFSGGGSGTDFTLKISRVEAEDLGVYFCFQSSL
VPLTFGAGTKLELK
21D9 Heavy
(SEQ ID NO: 48)
EVQLQQSGAELVRPGSSVKMSCKTSGYTFTTYGLNWVKQRPGQGLEWIG
YIYFGNGYTDYSEKFKGKATLTSDTSSSTAYMQLSSLTSEDSAIYFCAR
WVTGYYFDYWGQGTTLTVSS
21D9 Kappa
(SEQ ID NO: 49)
DVLMTQIPLSLPVSLGDQASISCRSSRTIVHSNGNTYLEWYLQKPGQSP
KLLIYKVSNRFSGVPDRFSGSGSGTDFTLKISRVEAEDLGVYYCFQSSL
VPPTFGGGTKLEI
7G4 Heavy
(SEQ ID NO: 50)
EVQLQQSGAELVRPGSSVKMSCKTSGYTFTNYGLNWVKQRPGQGLEWIG
YIYIGNGYTDYNENFKGKATLTSDTSSSTAYMQLSSLTSEDSAIYFCAR
WRPGYFFDYWGQGTTLTVSS
7G4 Kappa
(SEQ ID NO: 51)
DVLMTQTPLSLPVGLGDQASISCRSSQNIVHSNGDTYLEWYLLKPGQSP
QLLIYKVSNRFSGVPDRFSGGGSGTDFTLKISRVEAEDLGVYYCFQSSL
VPLTFGAGTKLELK
11H4 Heavy
(SEQ ID NO: 52)
EVQLQQSGAELVRPGSSVKMSCKTSGYTFTNYGVIWVKQRPGQGLEWIG
YIYIGNGFTDYSEKFKGKATLTSDTSSSTAYMHLISLTSEDSAIYFCGR
WRPGYYFDYWGQGTSLTVSS
11H4 Kappa
(SEQ ID NO: 53)
DVLMTQIPLSLPVSLGDQASISCRSSQNIVHSNGNTYLEWYLQKPGQSP
KLLIYKVSNRFSGVPDRFSGSGSGTDFTLKISRVEAEDLGVYYCFQSSL
VPLTFGAGTKLELK
13B12 Heavy
(SEQ ID NO: 54)
EVQLQQSGVELVRPGSSVKMSCKTSGYTFTNYGIIWVKQRPGQGLEWIG
YIYIGNGYTDCNEKFKGKASLTSDTSSSTAYMHLSSLTSEDSAIYFCGR
WRPGYYFDYWGQGTTLTVSS
13B12 Kappa
(SEQ ID NO: 55)
NVLMTQTPLSLPVSLGDQASISCRSSQTIVHSNGNTYLEWYLQKPGQSP
KLLIYKVSNRFSGVPERFSGSGSGTDFTLKISRVEAEDLGVYYCFQSSL
VPLTFGAGTKLELK
13H11 Heavy
(SEQ ID NO: 56)
EVQLQQSGVELVRPGSSVKMSCKTSGYTFTNYGIIWVKQRPGQGLEWIG
YISIGNGYTDCNEKFKGKASLTSDTSSSTAYMHLSSLTSEDSAIYFCGR
WRPGYYFDYWGQGTTLTVSS
13H11 Kappa
(SEQ ID NO: 57)
DVLMTQTPLSLPVSLGDQASISCRSSQTIVHSNGNTYLEWYLQKPGQSP
KLLIYKVSNRFSGVPERFSGSGSGTDFTLKISRVEAEDLGVYYCFQSSL
VPLTFGAGTKLELK
25B12 Heavy
(SEQ ID NO: 58)
EVQLQQSGAELVRPGSSVQMSCKTSGYTFTNYGINWVRQRPGQGLEWIG
YIYIGSGYTDYKEKFRGKATLTSDTSSSTAYMQLSSLTSEDSAIYFCAR
WVNGYYFDYWGQGTTLAVSS
25B12 Kappa
(SEQ ID NO: 59)
DVLMTQTPLSLPVSLGDQASISCRSSQSIIHSNGNTYLEWYLQKPGQSP
KLLIYKVSHRFSGVPDRFSGSGSGTDFSLKISRVEAEDLGVYYCFQSSL
VPPTFGGGTKLEI
5D8 Heavy
(SEQ ID NO: 60)
EVQLQQSGVELVRPGSSVKMSCKTSGYTFTNYGIIWVKQRPGQGLEWIG
YIYIGNGYTDCNEKFKGKASLTSDTSSSTAYMHLSSLTSEDSAIYFCGR
WRPGYYFDYWGQGTTLTVSS
5D8 Kappa
(SEQ ID NO: 61)
DVLMTQTPLSLPVSLGDQASISCRSSQTIVHSNGNTYLEWYLQKPGQSP
KLLIYKVSNRFSGVPERFSGSGSGTDFTLKISRVEAEDLGVYYCFQSSL
VPLTFGAGTKLELK
3E10 Heavy
(SEQ ID NO: 62)
EVQFQQSGAELVRPGSSVKMSCKTSGYTFTSYGINWVKQRPGQGLEWIG
YIYIGNGYTDYNEKFRGKATLTSDTSSSTAYMQLSGLTSEDSATYFCVR
WRPGYFFDYWGQGTTLTVSS
3E10 Kappa
(SEQ ID NO: 63)
DVLMTQTPLSLPVSLGDQASISCRSSQSIVHSNGNTYLEWYLQKPGQSP
KLLIYKVSNRFSGVPDRFSGSGSGTDFTLKISRVETEDLGVYYCFQTSL
VPLTFGAGTRLELK
11D5 Heavy
(SEQ ID NO: 64)
EVQLVESGGDLVKPGGSLKVSCAASGFTFSSYGMSWIRQTPDNTLEWVA
TISYGGTYTFYSDNVKGRFTISRDDAKNTLYLQMSSLKSEDTAMYYCSS
LWETWLAYWGQGTLVTVSA
11D5 Kappa
(SEQ ID NO: 65)
DVLMTQTPLSLPVSLGDQASISCRSSLSIVHSNGNTYLEWYLQKPGQSP
KLLIYRVSNRFSGVPDRFSGSGSGTDFTLKISRVEAEDLGVYYCFQGSH
IPPTFGGGTKLEI

Predicted heavy-chain and light-chain CDRs for Ac-K280 clones and Ac-K311 clones are provided in the following tables.

TABLE 2A
Ac-K280 Monoclonal Heavy-Chain CDRs

| | | SEQ | | SEQ | | SEQ |
|---|---|---|---|---|---|---|
| Clone | CDR1 | ID NO: | CDR2 | ID NO: | CDR3 | ID NO: |
| 19B6, 17G8, 25E5, | GLNIEDDY | 66 | IDPENGDP | 70 | ATRGL | 76 |
| 28H2, 24G12, 29C3 | | | | | | |
| 9G4, 12B9 | GFNIKDYY | 67 | IDPENGET | 71 | IRDFEV | 77 |
| 23B7 | GLNIKDDY | 68 | IDPENGDT | 72 | ATRGL | 76 |
| 6C1 | GLNIKDDY | 68 | IDPENDDT | 73 | ATRGV | 78 |
| 6F7 | GFNIKDYF | 69 | IDPEDGKT | 74 | IRDFEV | 77 |
| 30H12 | GLNIKDDY | 68 | IDPENGDN | 75 | TTRGL | 79 |
| 10C7 | GFNIKDYY | 67 | IDPENGET | 71 | FIRDFEV | 80 |

TABLE 2B
Ac-K280 Monoclonal Light-Chain CDRs

| | | SEQ ID | | | SEQ ID |
|---|---|---|---|---|---|
| Clone | CDR1 | NO: | CDR2 | CDR3 | NO: |
| 19B6, 17G8, 25E5, 28H2, | SSVSSSY | 81 | STS | HQYHRSPLT | 85 |
| 23B7, 24G12, 29C3 | | | | | |
| 9G4, 12B9, 10C7 | QNIVHGSGHTY | 82 | KVS | FQGSHVPLT | 86 |
| 6C1 | SSISSTF | 83 | RTS | HQYHRSPLT | 85 |
| 6F7 | QSIVHDNGHTY | 84 | KVS | FQGSHIPLT | 87 |
| 30H12 | SSVSSSY | 81 | STS | HQYRRSPLT | 88 |

TABLE 3A
Ac-K311 Monoclonal Heavy-Chain CDRs

| | | SEQ | | SEQ | | SEQ |
|---|---|---|---|---|---|---|
| Clone | CDR1 | ID NO: | CDR2 | ID NO: | CDR3 | ID NO: |
| 14G8, 15E8, | GYTFTNYG | 89 | IYIGNGYT | 93 | ARWRPGYFFDY | 99 |
| 7G4 | | | | | | |
| 21D9 | GYTFTTYG | 90 | IYFGNGYT | 94 | ARWVTGYYFDY | 100 |
| 11H4 | GYTFTNYG | 89 | IYIGNGFT | 95 | GRWRPGYYFDY | 101 |
| 13B12, 5D8 | GYTFTNYG | 89 | IYIGNGYT | 93 | GRWRPGYYFDY | 101 |
| 13H11 | GYTFTNYG | 89 | ISIGNGYT | 96 | GRWRPGYYFDY | 101 |
| 25B12 | GYTFTNYG | 89 | IYIGSGYT | 97 | ARWVNGYYFDY | 102 |
| 3E10 | GYTFTSYG | 91 | IYIGNGYT | 93 | VRWRPGYFFDY | 103 |
| 11D5 | GFTGSSYG | 92 | ISYGGTYT | 98 | SSLWETWLAY | 104 |

TABLE 3B
Ac-K311 Monoclonal Light-Chain CDRs

| | | SEQ ID | | | SEQ ID |
|---|---|---|---|---|---|
| Clone | CDR1 | NO: | CDR2 | CDR3 | NO: |
| 14G8, 7G4, 11H4 | QNIVHSNGDTY | 105 | KVS | FQSSLVPLT | 112 |
| 15E8 | QNIVHTNGDTY | 106 | KVS | FQSSLVPLT | 112 |
| 21D9 | RTIVHSNGNTY | 107 | KVS | FQSSLVPPT | 113 |
| 13B12, 13H11, 5D8 | QTIVHSNGNTY | 108 | KVS | FQSSLVPLT | 112 |
| 25B12 | QSIIHSNGNTY | 109 | KVS | FQSSLVPPT | 113 |
| 3E10 | QSIVHSNGNTY | 110 | KVS | FQTSLVPLT | 114 |
| 11D5 | LSIVHSNGNTY | 111 | RVS | FQGSHIPPT | 115 |

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. A monoclonal antibody or an antigen binding fragment thereof that specifically binds acetylated lysine 280 in human tau protein.

2. The monoclonal antibody or an antigen binding fragment thereof of claim 1, comprising a CDR1, CDR2 and/or a CDR3 sequence that is at least 90% identical to a sequence selected from SEQ ID NOs: 66 to 88.

3. The monoclonal antibody or antigen binding fragment thereof of claim 1, comprising a heavy chain or light chain sequence that is at least 90% identical to a sequence selected from SEQ ID NOs: 18 to 43.

4. A monoclonal antibody or an antigen binding fragment thereof that specifically binds acetylated lysine 311 in human tau protein.

5. The monoclonal antibody or an antigen binding fragment thereof of claim 4, comprising a CDR1, CDR2 and/or a CDR3 sequence that is at least 90% identical to a sequence selected from SEQ ID NOs: 89 to 115.

6. The monoclonal antibody or antigen binding fragment thereof of claim 4, comprising a heavy chain or light chain sequence that is at least 90% identical to a sequence selected from SEQ ID NOs: 44 to 65.

7. The monoclonal antibody or antigen binding fragment thereof of claim 1, being humanized and/or chimeric.

8. (canceled)

9. The monoclonal antibody or antigen binding fragment thereof of claim 1, being:
(i) an antibody fragment such as a Fab, a Fab′, a F(ab)′₂, a scFv, a Fv fragment, a nanobody, an intrabody, a VHH, or a minimal recognition unit;
(ii) a full-length immunoglobulin molecule; or
(iii) a non-immunoglobulin scaffold such as an affibody, an affilin molecule, an AdNectin, a lipocalin mutein, a DARPin, a Knottin, a Kunitz-type domain, an Avimer, a Tetranectin, or a trans-body.

10. The monoclonal antibody or antigen binding fragment thereof of claim 1, being monovalent or multivalent, wherein the monoclonal antibody or an antigen binding fragment thereof is optionally bispecific, preferably a diabody, a single-chain diabody, or a tandem scFv.

11. The monoclonal antibody or antigen binding fragment thereof of claim 1, being chemically or biologically modified, e.g., glycosylated, PEGylated, or HESylated.

12. (canceled)

13. An isolated nucleic acid molecule comprising a sequence encoding the monoclonal antibody or an antigen binding fragment thereof of claim 1.

14. A vector comprising the nucleic acid molecule of claim 13.

15. A host cell comprising the nucleic acid molecule of claim 13.

16. A pharmaceutical composition comprising the monoclonal antibody or antigen binding fragment thereof of claim 1; and further a pharmaceutically acceptable carrier, diluent, or excipient.

17-18. (canceled)

19. A method of treating, preventing, or delaying progression of a disease or condition associated with a tau protein acetylated at lysine 280 and/or lysine 311 in a subject in need thereof, the method comprising administering to the subject an effective amount of the pharmaceutical composition of claim 16, thereby treating the disease or condition.

20. A method of preventing or delaying progression of the normal aging process associated with a tau protein acetylated at lysine 280 and/or lysine 311 in a subject in need thereof, the method comprising administering to the subject an effective amount of the pharmaceutical composition of claim 16, thereby preventing or delaying progression of the normal aging process.

21. The method of claim 19, wherein the disease or condition associated with a tau protein acetylated at lysine 280 and/or lysine 311 is at least one of Alzheimer's disease, Parkinson's disease, Parkinson's disease with dementia, dementia with Lewy bodies, amyotrophic lateral sclerosis, Down's syndrome, corticobasal degeneration, frontal temporal dementia, Pick's disease, multisystem atrophy, progressive supranuclear palsy, inclusion body myositis, prion protein cerebral amyoid angiopathy, argyrophilic grain disease, tangle predominant dementia, chronic traumatic encephalopathy, and traumatic brain injury.

22-27. (canceled)

28. A method of detecting the presence of tau protein acetylated at lysine 280 and/or lysine 311 in a biological sample, the method comprising:
(i) contacting the biological sample with the monoclonal antibody or antigen binding fragment thereof of claim 1 under conditions permissive for specific binding of the monoclonal antibody or antigen binding fragment thereof to tau protein acetylated at lysine 280 and/or lysine 311, and
(ii) detecting whether a complex between the monoclonal antibody or antigen binding fragment thereof and tau protein acetylated at lysine 280 and/or lysine 311 is formed.

29. A method of diagnosing a disease associated with a tau protein acetylated at lysine 280 and/or lysine 311 in a subject, the method comprising:
(i) contacting a biological sample from the subject with the monoclonal antibody or antigen binding fragment thereof of claim 1 under conditions permissive for specific binding of the monoclonal antibody or antigen binding fragment thereof to tau protein acetylated at lysine 280 and/or lysine 311, and
(ii) detecting whether a complex between the monoclonal antibody or antigen binding fragment thereof and tau protein acetylated at lysine 280 and/or lysine 311 is formed; wherein detection of the complex indicates the presence of a disease associated with a tau protein acetylated at lysine 280 and/or lysine 311 in the subject.

30-32. (canceled)

33. A kit comprising the monoclonal antibody or antigen binding fragment thereof of claim 1 together with a packaged combination of reagents with instructions.
